(19) United States
(12) Patent Application Publication
(10) Pub. No.: US 2023/0285560 A1
(43) Pub. Date: Sep. 14, 2023

(54) CD200 BLOCKADE TO INCREASE THE ANTI-TUMOR ACTIVITY OF CYTOTOXIC T CELLS

(71) Applicant: The Board of Trustees of the Leland Stanford Junior University, Stanford, CA (US)

(72) Inventors: Maria Grazia Roncarolo, Menlo Park, CA (US); Alma-Martina Cepika, Stanford, CA (US); Molly Kathryn Javier Uyeda, Stanford, CA (US); Brandon Cieniewicz, Sunnyvale, CA (US)

(21) Appl. No.: 18/020,028

(22) PCT Filed: Aug. 12, 2021
(86) PCT No.: PCT/US2021/045771

§ 371 Date: Feb. 6, 2023

Related U.S. Application Data
(60) Provisional application No. 63/065,972, filed on Aug. 14, 2020.

(51) Int. Cl. A61K 39/00; A61K 39/395; A61P 35/02

(52) U.S. Cl. CPC A61K 39/4635; A61K 39/4613; A61K 39/4611; A61K 39/3955; A61P 35/02

(57) ABSTRACT

Methods, compositions and kits for enhancing cell mediated killing of cancer cells are provided. Methods to enhance killing of such cancers, e.g. pAML, comprise administering an effective dose of a CD200 blocking agent in combination with an effective dose of cytotoxic immune cells.

### CROSS REFERENCE TO RELATED APPLICATION

The present application claims the benefit of and priority to U.S. Provisional Patent Application No. 63/065,972 filed Aug. 14, 2020, the entire disclosure of which is hereby.

### BACKGROUND

Pediatric AML affects over 700 children in the US every year. While pediatric AML (pAML) comprises only 25% of all pediatric acute leukemias, it accounts for almost half of pediatric leukemia-related deaths. Five-year survival rates for pAML have risen to over 60%, in part due to improved risk-stratification, supportive care, and post-relapse treatment. However, between 30-55% of patients eventually relapse, and relapse remains the most frequent cause of death. Current treatment for relapsed or treatment-refractory pAML is allogeneic hematopoietic stem cell transplantation (allo-HSCT). Unfortunately, allo-HSCT carries the significant risk of inducing life-threatening graft versus host disease (GvHD) mediated by donor-derived T cells. GvHD is the major cause of transplant-related morbidity and mortality, and the second leading cause of death in AML patients. GvHD can be treated with immunosuppressive drugs, but these treatments also impair donor-derived cells from clearing residual leukemia (GvL), thereby increasing the risk of relapse. Thus, new treatments that preserve GvL while preventing GvHD are urgently needed.

CD200, a type-I membrane glycoprotein, is expressed in a variety of cell types including T and B lymphocytes. Its receptor, CD200R is found on T, B, NK cells and myeloid cells. CD200 has been identified as a prognostic factor in acute myeloid leukemia and is found in many other hematological and non-hematological malignancies.

### SUMMARY

Compositions and methods are provided for enhanced NK or T cell killing of cancer, i.e. killing of cancer cells by a cytoxic immune cell. In some embodiments the cancer is a leukemia. In some embodiments the leukemia is a myeloid leukemia. In some embodiments the myeloid leukemia is acute myeloid leukemia (AML), including pediatric AML (pAML). In some embodiments the myeloid leukemia is Juvenile myelomonocytic leukemia (JMML). It is shown herein that expression of CD200 on primary acute myeloid leukemia blasts correlates with blast resistance to cytotoxic cell killing, inhibiting degranulation of cytotoxic T cells, and leading to reduced killing. Methods are provided herein to enhance killing of such cancers, e.g. pAML, JMML by administering an effective dose of a CD200 blocking agent in combination with an effective dose of cytotoxic immune cells.

In treatment of cancer with an effective dose of cytotoxic immune cells, e.g. cytotoxic T cell, the T cells can be provided in combination with an effective dose of an agent that blocks CD200 from interacting with its receptor expressed on T cells, including without limitation CD200R1, where the dose is effective to reduce inhibition of cytotoxic T cell killing relative to administration with the CD200 blocking agent. Agents for this purpose include antibodies, peptides, soluble receptor, small molecules, and the like. Antibodies may specifically bind to CD200, or to a CD200 receptor, e.g. CD200R1. An alternative agent may bind to both as a bispecific agent. Alternatively T cells can be engineered to reduce or ablate expression of a CD200 receptor, e.g. by anti-sense RNA, RNAi, CRISPR engineering to knock out the receptor gene, and the like.

Cytotoxic T cells can be pre-treated with an effective dose of an agent that binds to a CD200 receptor, e.g. an antibody that binds to CD200 receptor, prior to administration of the cytotoxic T cells to a patient for treatment of cancer. In such embodiments the T cells can be pre-incubated with the agent for a period of time sufficient to block the CD200 receptor, e.g. for a period of up to 1 day prior to administration, up to 12 hours prior to administration, up to 6 hours, up to 3 hours, up to 1 hour, or immediately prior to administration.

Patients for treatment of a leukemia with an effective dose of cytotoxic T cells can be pre-treated with an effective dose of an agent that binds to CD200, e.g. an antibody that binds to CD200, prior to administration of the cytotoxic T cells to a patient for treatment of cancer. The patient is, in some embodiments, a pAML patient or a JMML patient. An an effective dose of an agent can be administered with the effective dose of cytotoxic T cells, e.g. for a period of up to 3 days prior to administration of cytotoxic T cells, up to 1 day prior to administration, up to 12 hours prior to administration, up to 6 hours, up to 3 hours, up to 1 hour, immediately prior to administration; or can be administered concurrently with cytotoxic T cell administration.

A cancer sample, e.g. a pAML sample, from a patient may be evaluated for expression of CD200 on the cancer cells prior to treatment. A cancer sample for this purpose is usually a hematopoietic sample, e.g. blood, bone marrow, etc. In some embodiments, the presence of cancer cells, e.g. AML blast cells present in a blood sample, that express CD200 indicates a need to administer a CD200 blocking agent in combination with cytotoxic T cell therapy. A population determined to be CD200 positive may be at least about 0.01% positive, at least about 0.1% positive, at least 1% positive, at least 10% positive, or more, of the blast cell population in a blood or bone marrow sample.

In some embodiments the cytotoxic T cell for treatment of cancer, e.g. pAML, is an engineered CD4+ T cell that expresses IL-10, which cells may be referred to as LV-10 cells. LV-10 cells may be allogeneic or autologous with respect to the cancer patient for treatment. LV-10 cells are used, without limitation, in the treatment of AML, e.g. pediatric AML.

In some embodiments administration of an effective dose of LV-10 cells for treatment of cancer, e.g. pAML, is performed in combination with allogeneic hematopoietic stem cell transplantation (allo-HSCT). Alternatively, administration of an effective dose of LV-10 cells for treatment of cancer, e.g. pAML, are administered in the absence of allo-HSCT, where the LV-10 cells provide for a GvL effect, e.g. when the patients' own immune cells are depleted. In some embodiments, administration of an effective dose of LV-10 cells for treatment of cancer, e.g. pAML, is used as an alternative to induction chemotherapy, prior to allo-HSCT.

In other embodiments a cytotoxic T cell for the treatment of cancer, e.g. pAML, in the methods disclosed herein is a CD8+ T cell. A cytotoxic CD8+ T cell may be allogeneic or autologous with respect to the cancer patient for treatment. A population of cytotoxic CD8⁺ T cells is usually expanded in vitro prior to administering to a cancer patient. In other embodiments the cytotoxic immune cell is an NK, NKT, or iNKT cell.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: Pediatric AML have 3 levels of sensitivity to killing by LV-10 cells. (A) Primary pAML bone marrow aspirates were co-cultured at a 1:1 ratio with LV-10 cells. After 4d, residual pAML (CD3⁻) were enumerated by flow cytometry (killing assay). Elimination efficiency (E.E.) was calculated for each LV-10 using the equation 1−(AML remaining in LV-10 co-culture/AML remaining alone). U937 and K562 cells were included as positive and negative controls, respectively, for killing. The solid line indicates the median elimination efficiency, while the box boundaries indicate the range. Each dot represents the E.E. determined by co-culture with one LV-10 cell line, N=2-4. (B) Left panel contains representative plots of remaining pAML after the killing assay with or without LV-10; numbers on plots indicate the number of pAML cells normalized to the CountBright beads. Gating is set based on AML cells cultured alone. Right panel graphs the representative examples to show the absolute differences between the number of pAML cells cultured with LV-10 and alone.

FIG. 2: Sensitive and resistant pAML have distinct gene expression profiles. (A) Two-dimensional heatmap of differentially expressed genes (DEG identified between sensitive (S) and resistant (R) pAML. DEGs with FDR<0.05 and abs(Log 2FC) 2 were identified using DESeq2. Gene expression values were normalized with respect to the sensitive pAML group, by subtracting the mean expression of sensitive pAML. (B) Gene set enrichment in sensitive pAML. GO term enrichment was performed using Gene Set Enrichment Analysis and GO terms with FDR q<0.2 were visualized using EnrichmentMap in Cytoscape. Circle size is inversely scaled to the FDR q-val. (C) Sensitive pAML have high expression of mature myeloid markers. Top panels show bulk RNA-seq log 2 counts, and the bottom panels show the frequency of pAML expressing the indicated proteins, measured using clinical flow cytometry gated on blast cells. Error bars: median and interquartile range. *=p<0.05. IR=Intermediate Resistant.

FIG. 3: Sensitive and resistant pAML signatures group TARGET pAML into 3 clusters. Euclidean clustering of the DEGs between sensitive and resistant pAML (FDR<0.05, and abs(Log 2FC)≥2) detected in sequencing data from our Stanford dataset and the TARGET pAML dataset. Measured LV-10 killing sensitivity, risk group, and FAB diagnosis are matched to each pAML when applicable. The TARGET dataset does not have associated LV-10 killing assay outcomes. Expression color is scaled per gene row. ND=not determined.

FIG. 4: CD200 expression is upregulated in resistant pAML and can impair LV-10-mediated degranulation and cytotoxicity. (A) Expression of 395 genes positively or negatively correlating with pAML sensitivity. The Spearman correlation of the expression of each gene to the median elimination efficiency (E.E.) of each pAML was calculated and plotted with genes represented as bars. 2181 genes had a correlation with p<0.05, 395 of which had an abs(R) 0.7 (red bars). (B) Data-mining strategy to identify genes conferring pAML resistance to LV-10 killing. Genes expressed 4-fold or more in the resistant pAML from the DEG analysis between sensitive vs resistant and the list of genes negatively correlated with E.E. with p<0.05 and R≤−0.07 were used to identify overlap in a Venn diagram. Genes appearing in both enriched in resistant and negatively correlated with E.E. were overlaid with genes encoding surface proteins identified in the Cell Surface Protein Atlas, resulting in 10 pAML genes encoding surface proteins. These genes were manually annotated for potential interaction with T cell surface proteins, identifying CD200. (C) CD200 gene expression in pAML blasts; log 2 counts. Error bars: median and interquartile range. (D) CD200 protein expression on pAML, flow cytometry. Left panel: representative plots for one sensitive and one resistant pAML blast; right panel: cumulative data. Values from R and IR pAML are grouped, with IR pAML in grey. Line represents mean. (E) CD200R1 is expressed on both LV-10 and LV-GFP cells. CD200R1 expression was measured in the CD3⁺CD4⁺NGFR⁺ population by flow cytometry. (N=8). Line represents mean; error bars SD. (F) CD200 overexpression impairs LV-10 degranulation. LV-10 were co-cultured with target myeloid tumor cell lines at a 10:1 E:T ratio for 6 h. LV-10 cell line (N=7) degranulation, as measured by CD107a⁺granzyme B⁺ co-expression, was determined in co-culture with untransduced U937 and ALL-CM cells (No Vector), sorted GFP⁺ U937 and ALL-CM cells transduced with an empty vector (Empty Vector), or sorted GFP⁺CD200⁺ U937 and ALL-CM cells transduced with CD200 (CD200 Vector). *=p<0.05, **=p<0.01, Friedman ANOVA with Dunn's post hoc test. (G) Cumulative CD107a+GZMB+% degranulation of LV-10 against U937 overexpressing CD200 when pre-treated with isotype control or anti-CD200R1 blocking antibody prior to co-culture (N=6) *=p<0.05, Wilcoxon test. (H) CD200 reduces LV-10-mediated killing. LV-10 cells were co-cultured with empty vector- or CD200-overexpressing U937 or ALL-CM cells at a 1:1 E:T ratio for 3 d. Surviving cells were enumerated by flow cytometry. Ratio of remaining pAML was calculated as: (#remaining cells in CD200-overexpressing AML+LV-10/#remaining cells in CD200-overexpressing AML alone)/(#remaining cells in empty vector−AML+LV-10/#remaining cells in empty vector AML alone). (N=8, *=p<0.05, Wilcoxon test.) GZMB=Granzyme B.

FIG. 5: LV-10 cells are produced with high efficiency and have Tr1 functionality. (A) LV-10 transduction efficiency. NGFR expression in CD4⁺ cells was measured 6 days after transduction by flow cytometry to determine transduction efficiency. (N=7). (B) Transduced cell lines retain purity in culture. The level of NGFR⁺ or GFP⁺NGFR⁺ cells was measured after 2 feeder cycles by flow cytometry. (N=7). (C) LV-10 cells have high IL-10 and IFNγ expression. Cytokine secretion from LV-10 and LV-GFP cells was measured at the end of a feeder cycle. 100,000 cells were cultured for 48 h with 10 μg/ml plate-bound anti-CD3 and 1 μg/ml soluble anti-CD28. Cytokine secretion was measured by ELISA. (N=7) (*=p<0.05, **=p<0.01, Wilcoxon Test.) (D) LV-10 have high IL-10/IL-4 expression ratios. The IL-10 to IL-4 expression ratio was calculated for LV-10 and LV-GFP cells after 48 h stimulation with 10 μg/ml plate-bound anti-CD3 and 1 μg/ml soluble anti-CD28. (N=7). (*=p<0.05, Wilcoxon test.) (E) LV-10 cells have higher baseline granzyme B expression. LV-10 or LV-GFP cells were fixed and stained for the presence of intracellular granzyme B. Overall percentages of granzyme B⁺ cells are shown. (N=12, significance indicated by Wilcoxon Test.) (F) LV-10 cells have superior degranulation against myeloid target cells. LV-10 and LV-GFP cells were incubated alone or with target cells at a 10:1 E:T ratio in the presence of anti-CD107a antibody. After 6 h, expression of granzyme B and CD107a were measured by flow cytometry. Overall CD107a⁺ Granzyme B⁺ cells are shown for cells co-stimulated with K562, ALL-CM, or U937 cells. (N=10-12, significance indicated by Wilcoxon Test.) Representative plots for LV-10 and LV-GFP at baseline or after co-culture with U937 and K562 are shown on the right. (G) LV-10 cells efficiently eliminate the control myeloid U937 and ALL-CM cell lines but not the erythroleukemic K562 cell line. U937 or K562 cells were co-cultured at a 1:1 E:T ratio with LV-10 or LV-GFP cells. After 3 d, remaining target cells (CD3-NGFR⁻) were enumerated using flow cytometry. Results for 4 independently derived LV-10 and LV-GFP pairs are shown. Elimination efficiency (E.E) was calculated as 1−(number of remaining target cells with LV-10 or LV-GFP)/(number of remaining target cells alone). (N=7 per cell line.) Representative plots for U937 or K562 cells alone or with LV-10 or LV-GFP are shown on the right. GZMB=Granzyme B

FIG. 6: pAML sensitivity does not correlate with survival in culture or blast percentage. (A) pAML were cultured alone in media supplemented with 20 ng/ml IL-3 and G-CSF. At the end of the 4 d culture, remaining pAML were enumerated by flow cytometry and the percentage remaining of the plated pAML was determined. pAML survival in culture was graphed against the average elimination efficiency measured for that pAML (FIG. 1), and a linear regression curve was derived. (N=23) (B) Blast percentage in pAML does not correlate with killing. Blast percentage, when available, was obtained by clinical laboratory analysis of patient's bone marrow aspirate, for each pAML was graphed against the median elimination efficiency measured for that pAML (FIG. 1), and a linear regression curve was derived. (N=21)

FIG. 7: DEGs between intermediate resistant pAML and sensitive or resistant groups. (A) Two-dimensional heatmap of DEGs identified between sensitive and intermediate resistant pAML or (B) intermediate resistant and resistant pAML. 247 genes were differentially expressed between sensitive and intermediate resistant pAML, and 27 genes between intermediate resistant and resistant with FDR<0.05, and abs(Log₂FC)≥2. Gene expression values were normalized to the average expression in sensitive pAML by subtracting the mean expression of sensitive pAML.

FIG. 8: Principle component analysis (PCA) of clinical lab flow cytometry. The frequency of 28 different proteins was measured on pAML gated for blasts by the Bass Center's clinical flow cytometry lab and was used to perform PCA analysis. Each dot represents one pAML patient sample. Labeled arrows represent the top 10 variable loadings in PC1 and arrow lengths are scaled by their contribution to the variance explained %.

FIG. 9: pAML origin is not a dominant technical covariate. Our 14 sequenced Stanford pAML were reanalyzed to match the analysis of the 187 RNA sequenced TARGET pAML dataset. (A) The top 10% most variably expressed genes between all 201 pAML were identified and used to perform PCA analysis of all pAML. (B) Expression of the top 10% most variably expressed genes clusters Stanford with TARGET pAML. Expression of the top 10% most variable genes was visualized as a two-dimensional heatmap. Sensitivity to elimination, risk group stratification, the timepoint the sample was acquired, and FAB category are also displayed for each pAML when applicable. Expression color is scaled per gene row.

FIG. 10: CD200 overexpression in U937 and ALL-CM cell lines. (A) Schematic of lentiviral CD200 overexpression plasmid. CD200 cDNA was ligated into pLVX-IRES-ZsGreen1 to enable bicistronic expression of CD200 and ZsGreen1 GFP. (B) CD200 overexpression in ALL-CM and U937 myeloid cell lines. ALL-CM and U937 were transduced with concentrated CD200 or empty vector lentivirus. After 5 d, cells were stained for CD200 and analyzed by flow cytometry.

FIG. 11: Degranulation response of LV-10 against U937-CD200 cells can be partially blocked by anti-CD200R1 antibody treatment. (A) LV-10 were treated with isotype control or anti-CD200R1 blocking antibody prior to co-culture with targets. Antibody treated LV-10 cells were seeded at 10:1 effector:target (E:T) ratio and co-cultured with U937 WT or U937-CD200 overexpressing cells. Representative CD107a vs GranzymeB plots from one LV-10 donor are shown. Cells are gated on lymphocytes/live cells/CD3⁺/CD4⁺. (B) Cumulative CD107a⁺GranzymeB⁺% degranulation of LV-10 against U937-WT when treated with 25 ug/ml isotype control or anti-CD200R1 blocking antibody (N=6). Statistical analysis performed with Wilcoxon test. (C) LV-10 cells were treated for 30 min with 25 or 50 ug/ml anti-CD200R1 blocking antibody. In the left panel, representative histograms show CD200R1 surface staining. On the right panel, CD200R1 staining reduction was calculated by dividing the CD200R1 APC MFI or % CD200R1⁺ of antibody-treated (red) sample by the respective value in the untreated (black) sample. (blue: LV-GFP donor1, red: LV-10 donor1; pink: LV-GFP donor2, orange: LV-10 donor2; green: LV-GFP donor3, black: LV-10 donor3)

FIG. 12: CD200 overexpression in U937 and ALL-CM cell lines reduces degranulation and cytotoxicity of LV-GFP cells. (A) LV-GFP cell line (N=8) degranulation, as measured by CD107a⁺granzyme B⁺ co-expression, was determined in co-culture with untransduced U937 and ALL-CM cells (No Vector), sorted GFP⁺ U937 and ALL-CM cells transduced with an empty vector (Empty Vector), or sorted GFP⁺CD200⁺ U937 and ALL-CM cells transduced with CD200 (CD200 Vector). (*=p<0.05, **=p<0.01, ***=p<0.001. Friedman ANOVA with Dunn's post hoc test.) (B) LV-GFP cells were co-cultured with empty vector or CD200-overexpressing U937 or ALL-CM cells at a 1:1 E:T ratio for 3 d. Surviving cells were enumerated by flow cytometry. Ratio of remaining pAML was calculated as: (#remaining cells in CD200-overexpressing AML+LV-GFP/#remaining cells in CD200-overexpressing AML alone)/(#remaining cells in empty vector−AML+LV-GFP/#remaining cells in empty vector AML alone) (N=8, **=p<0.01, Wilcoxon test.)

FIG. 13. JMML sensitivity to killing and CD200 expression. A. Elimination efficiency of 8 primary JMML samples co-cultured with LV-10 cells (i.e. the killing assay, performed equivalently as with pediatric AML samples shown on FIG. 1), in comparison to negative control cell line K562 (resistant to LV-10 killing) and positive control cell line (U937, sensitive to LV-10 killing). Samples were tested in triplicates when available. JMML donor IDs start with either JSP or JMML. B. Flow cytometry staining of CD200 protein expression on five JMML patient samples, designated as lineage-negative (Lin⁻) and CD34⁺ cells. Gating was performed according to the unstained Lin⁻ control cells. C. Frequency of CD200⁺ JMML samples in comparison to pediatric AML samples (from FIG. 4B). Kruskal Wallis ANOVA, p=0.0097, with Dunn's post hoc test; adjusted p value*<0.05.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

CD200. OX-2 membrane glycoprotein, also named CD200 (Cluster of Differentiation 200) is a type-1 membrane glycoprotein, which contains two immunoglobulin domains, and thus belongs to the immunoglobulin superfamily. Studies of the related genes in mouse and rat suggest that this gene may regulate myeloid cell activity and delivers an inhibitory signal for the macrophage lineage in diverse tissues. Multiple alternatively spliced transcript variants that encode different isoforms have been found for this gene. Reference sequences for the human proteins include NP_001004196, NP_001305755, NP_001305757, NP_001305759, NP_005935.

CD200R1 is an Ig superfamily transmembrane glycoprotein expressed on the surface of myeloid cells; it can also be induced in certain T-cell subsets. CD200R1 interacts with CD200, which is also an Ig superfamily transmembrane glycoprotein, to downregulate myeloid cell functions. CD200 is expressed on the surface of a variety of cells including neurons, epithelial cells, endothelial cells, fibroblasts, lymphoid cells, and astrocytes. The regulation of CD200R1 signaling can occur by posttranslational modification, e.g. phosphorylation of tyrosines in the CD200R1 cytoplasmic tail, or by the inducible expression or downregulation of either CD200R1 or CD200. Each of these mechanisms can ultimately be exploited by pathogens.

Unlike most immune inhibitory receptors, CD200R1 does not contain an ITIM. Instead, human CD200R1 contains three cytoplasmic tyrosine residues, Y291, Y294, and Y302, one of which, Y302/Y297, is located within a phosphotyrosine binding (PTB) domain recognition motif (NPxY). Stimulation by CD200 leads to the phosphorylation of these tyrosines by Src kinases, which recruit the adapter protein downstream of tyrosine kinase (Dok) 2 through its PTB domain. Y302/Y297 and to a lesser extent Y291/Y286 are the major tyrosine residues required for CD200R1 association with Dok2. Dok2 serves as the major initiator of signaling through CD200R1, beginning with binding to Ras-GTPase activating protein (RasGAP) and is required for CD200R1 function. This is in contrast to ITIM containing inhibitory receptors, which utilize SHPs and SHIP-1 as the major initiator proteins and Dok proteins as secondary modulators of downstream signaling.

Anti-CD200 agent. As used herein, the term “anti-CD200 agent” refers to any agent that reduces the binding of CD200 (e.g., on a target cell) to CD200R1 (e.g., on a T cell). Non-limiting examples of suitable anti-CD200 reagents include CD200R1 soluble polypeptides, anti-CD200R1 antibodies, soluble CD200 polypeptides, and anti-CD200 antibodies or antibody fragments. In some embodiments, a suitable anti-CD200 agent specifically binds CD200 to reduce the binding of CD200 to CD200R1. In some embodiments, a suitable anti-CD200 agent specifically binds CD200R1 to reduce the binding of CD200 to CD200R1. A suitable anti-CD200 agent that binds CD200R1 does not activate CD200R1 (e.g., in the CD200R1-expressing T cell). The efficacy of a suitable anti-CD200 agent can be assessed by assaying the agent. In an exemplary assay, target cells are incubated in the presence or absence of the candidate agent. An agent for use in the methods of the invention will upregulate T cell or NK cell-mediated degranulation, e.g. release of perforin or granzymes by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to the level in the absence of the agent.

A CD200R1 a reagent comprises the portion of CD200R1 that is sufficient to bind CD200 at a recognizable affinity, which normally lies between the signal sequence and the transmembrane domain, or a fragment thereof that retains the binding activity. A suitable CD200R1 reagent reduces (e.g., blocks, prevents, etc.) the interaction between the native proteins CD200R1 and CD200. In some embodiments, a CD200R1 reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. In other embodiments, the second polypeptide is any suitable polypeptide that is substantially similar to Fc, e.g., providing increased size, multimerization domains, and/or additional binding or interaction with Ig molecules.

Anti-CD200 antibodies. In some embodiments, a subject anti-CD200 agent is an antibody that specifically binds CD200 (i.e., an anti-CD200 antibody) and reduces the interaction between CD200 on one cell (e.g., a cancer cell) and CD200R1 on another cell (e.g., a T cell). In some embodiments, a suitable anti-CD200 antibody does not activate CD200 upon binding. Suitable anti-CD200 antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

For example, Samalizumab is a recombinant humanized monoclonal antibody that targets CD200, an immunoregulatory cell surface member of the immunoglobulin superfamily that dampens excessive immune responses and maintains self-tolerance. Anti-CD200-blocking antibody (TTI-CD200) is a fully human antibody that neutralises human CD200 with nanomolar potency. MRC OX-104 monoclonal antibody specifically binds to CD200. Anti-CD200 antibodies are disclosed in Kretz-Rommel et al. (2007) J Immunol 178 (9) 5595-5605; etc.

Anti-CD200R1 antibodies. In some embodiments, a subject anti-CD200 agent is an antibody that specifically binds CD200R1 (i.e., an anti-CD200R1 antibody) and reduces the interaction between CD200 on one cell and CD200R1a on another cell. A suitable anti-CD200R1 antibody specifically binds CD200R1 without activating/stimulating enough of a signaling response to inhibit cytoxicity) and blocks an interaction between CD200R1 and CD200. Suitable anti-CD200R1a antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

An antibody that binds to an antigen of interest, is one that binds the antigen with sufficient affinity such that the antibody or binding molecule is useful as a diagnostic and/or therapeutic agent in targeting the antigen, and does not significantly cross-react with other proteins. In such embodiments, the extent of binding of the antibody or other binding molecule to a non-targeted antigen will usually be no more than 10% as determined by fluorescence activated cell sorting (FACS) analysis or radioimmunoprecipitation (RIA).

The terms “specific binding,” “specifically binds,” and the like, refer to non-covalent or covalent preferential binding to a molecule relative to other molecules or moieties in a solution or reaction. In some embodiments, the affinity of one molecule for another molecule to which it specifically binds is characterized by a KD (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less). “Affinity” refers to the strength of binding, increased binding affinity being correlated with a lower KD. In an embodiment, affinity is determined by surface plasmon resonance (SPR), e.g. as used by Biacore systems. The affinity of one molecule for another molecule is determined by measuring the binding kinetics of the interaction, e.g. at 25° C.

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region, although as well be discussed here, a variety of non-natural configurations of antibodies are known and used in the art.

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or enzymatic activity. A functional antibody or other binding molecule may also block ligand activation of a receptor or act as an agonist or antagonist. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include 3 CDR sequences, which may be obtained from available antibodies with the desired specificity, or may be obtained from antibodies developed for this purpose. One of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139., each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis (ADCP); and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called κ and λ, based on the amino acid sequences of their constant domains.

A “functional Fc region” possesses an “effector function” of a native-sequence Fc region. Exemplary effector functions include C1q binding; CDC; Fc-receptor binding; ADCC; ADCP; down-regulation of cell-surface receptors (e.g., B-cell receptor), etc. Such effector functions generally require the Fc region to be interact with a receptor, e.g. the FcγRI; FcγRIIA; FcγRIIB1; FcγRIIB2; FcγRIIIA; FcγRIIIB receptors, and the low affinity FcRn receptor; and can be assessed using various assays as disclosed, for example, in definitions herein. A “dead” Fc is one that has been mutagenized to retain activity with respect to, for example, prolonging serum half-life, but which does not activate a high affinity Fc receptor. An Fc may also have decreased binding to complement.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). Other Fc variants are possible, including without limitation one in which a region capable of forming a disulfide bond is deleted, or in which certain amino acid residues are eliminated at the N-terminal end of a native Fc form or a methionine residue is added thereto. Thus, in one embodiment of the invention, one or more Fc portions of the scFc molecule can comprise one or more mutations in the hinge region to eliminate disulfide bonding. In yet another embodiment, the hinge region of an Fc can be removed entirely. In still another embodiment, the molecule can comprise an Fc variant.

Further, an Fc variant can be constructed to remove or substantially reduce effector functions by substituting, deleting or adding amino acid residues to effect complement binding or Fc receptor binding. For example, and not limitation, a deletion may occur in a complement-binding site, such as a C1q-binding site. Techniques of preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g. Pichia Pastoris, and mammalians cell line, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See e.g., WO 07/117505). The presence of particular glycosylation moieties has been shown to alter the function of Immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See e.g., Kaneko, et al, Science 313:760 (2006)), whereas removal of fucose from the IgG leads to enhanced ADCC activity (See e.g., Shoj-Hosaka, et al, J. Biochem., 140:777 (2006)).

In alternative embodiments, antibodies of the invention may have an Fc sequence with enhanced effector functions, e.g. by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into >50-fold higher ADCC activity compared with wild type IgG1 controls. Protein engineering, through amino acid mutations in the Fc portion of IgG1, has generated multiple variants that increase the affinity of Fc binding to FcγRIIIA. Notably, the triple alanine mutant S298A/E333A/K334A displays 2-fold increase binding to FcγRIIIA and ADCC function. S239D/I332E (2×) and S239D/1332E/A330L (3×) variants have a significant increase in binding affinity to FcγRIIIA and augmentation of ADCC capacity in vitro and in vivo. Other Fc variants identified by yeast display also showed the improved binding to FcγRIIIA and enhanced tumor cell killing in mouse xenograft models. See, for example Liu et al. (2014) JBC 289(6):3571-90, herein specifically incorporated by reference.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

“Fv” is the minimum antibody fragment, which contains a complete antigen-recognition and antigen-binding site. The CD3 binding antibodies of the invention comprise a dimer of one heavy chain and one light chain variable domain in tight, non-covalent association; however additional antibodies, e.g. for use in a multi-specific configuration, may comprise a VH in the absence of a VL sequence. Even a single variable domain (or half of an Fv comprising only three hypervariable regions specific for an antigen) has the ability to recognize and bind antigen, although the affinity may be lower than that of two domain binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

“Humanized” forms of non-human (e.g., rodent) antibodies, including single chain antibodies, are chimeric antibodies (including single chain antibodies) that contain minimal sequence derived from non-human immunoglobulin. See, for example, Jones et al, (1986) Nature 321:522-525; Chothia et al (1989) Nature 342:877; Riechmann et al (1992) J. Mol. Biol. 224, 487-499; Foote and Winter, (1992) J. Mol. Biol. 224:487-499; Presta et al (1993) J. Immunol. 151, 2623-2632; Werther et al (1996) J. Immunol. Methods 157:4986-4995; and Presta et al (2001) Thromb. Haemost. 85:379-389. For further details, see U.S. Pat. Nos. 5,225,539; 6,548,640; 6,982,321; 5,585,089; 5,693,761; 6,407,213; Jones et al (1986) Nature, 321:522-525; and Riechmann et al (1988) Nature 332:323-329.

Acute Myelocytic Leukemia (AML, Acute Myelogenous Leukemia; Acute Myeloid Leukemia). In AML, malignant transformation and uncontrolled proliferation of an abnormally differentiated, long-lived myeloid progenitor cell results in high circulating numbers of immature blood forms and replacement of normal marrow by malignant cells. Symptoms include fatigue, pallor, easy bruising and bleeding, fever, and infection; symptoms of leukemic infiltration are present in only about 5% of patients (often as skin manifestations). Examination of peripheral blood smear and bone marrow is diagnostic. Treatment includes induction chemotherapy to achieve remission and post-remission chemotherapy (with or without stem cell transplantation) to avoid relapse.

AML has a number of subtypes that are distinguished from each other by morphology, immunophenotype, genetic abnormalities, and cytochemistry. Described classes include, based on predominant cell type, including myeloid, myeloid-monocytic, monocytic, erythroid, and megakaryocytic. Subtypes include Core Binding Factor leukemias, acute promyelocytic leukemia, etc.

Remission induction rates range from 50 to 85%. Long-term disease-free survival reportedly occurs in 20 to 40% of patients and increases to 40 to 50% in younger patients treated with haematopoetic stem cell transplantation.

Prognostic factors help determine treatment protocol and intensity; patients with strongly negative prognostic features are usually given more intense forms of therapy, because the potential benefits are thought to justify the increased treatment toxicity. The most important prognostic factor is the leukemia cell karyotype; favorable karyotypes include t(15;17), t(8;21), and inv16 (p13;q22). Negative factors include increasing age, a preceding myelodysplastic phase, secondary leukemia, high WBC count, and absence of Auer rods. The FAB or WHO classification alone does not predict response.

AML responds to few induction regimens designed to induce remission. The basic induction regimen includes cytarabine by continuous IV infusion or high doses for 5 to 7 days; daunorubicin or idarubicin is given IV for 3 days during this time. Some regimens include 6-thioguanine, etoposide, vincristine, and prednisone, but their contribution is unclear. Treatment usually results in significant myelosuppression, with infection or bleeding; there is significant latency before marrow recovery. During this time, meticulous preventive and supportive care is vital.

Pediatric AML. The incidence of AML in infants is 1.5 per 100,000 individuals per year, the incidence decreases to 0.9 per 100,000 individuals aged 1-4 and 0.4 per 100,000 individuals aged 5-9 years, after which it gradually increases into adulthood, up to an incidence of 16.2 per 100,000 individuals aged over 65 years.

The underlying cause of AML is unknown, and childhood AML generally occurs de novo. In adult and elderly patients, AML is often preceded by myelodysplastic syndrome (MDS), but in children, the occurrence of AML preceded by clonal evolution of preleukemic myeloproliferative diseases is rare. Germline affected individuals, such as those with Fanconi anemia or Bloom syndrome, have an increased risk for developing AML as a secondary malignancy. Germ-line mutations in several genes, such as TP53, RUNX1, GATA2 and CEBPA, have been found in families with an unexplained high risk of AML, suggesting a familial predisposition to develop AML. Some types of AML develop from specific causes, e.g. secondary AML (sAML). Therapy-related AML (t-AML) and AML with myelodysplasia-related changes (AML-MRC) are types of sAML. Roughly one-third of all AML cases are diagnosed as either t-AML or AML-MRC

Risk-group stratification is usually based on (cyto)genetic abnormalities present in the leukemic blasts in combination with early response to treatment, either specified as complete remission (CR) rate after one or two courses or applying minimal-residual disease measurements. The chemotherapeutic regimens consist of 4-5 cycles of intensive chemotherapy, typically including cytarabine combined with an anthracycline.

Juvenile myelomonocytic leukemia (JMML) is a myelodysplastic (MDS)/myeloproliferative neoplasm (MPN) overlap syndrome of the pediatric age group characterized by sustained, abnormal, and excessive production of myeloid progenitors and monocytes, aggressive clinical course, and poor outcomes. Unlike acute leukemias, there is no maturation arrest in myeloid differentiation; hence the number of blasts in the peripheral blood (PB) or bone marrow (BM) may be low even in the presence of a high total leukocyte count (TLC). The differentiation pathway is shunted towards the monocytic differentiation and the progenitor colonies of JMML cells show a spectrum of differentiation, including blasts, pro-monocytes, monocytes, and macrophages. The progenitor cells in JMML show high sensitivity to G-CSF in-vitro. The overproduction of the myeloid lineage cells leads to a suppression of other cell lines; consequently, these patients can present with anemia and thrombocytopenia. JMML presents in infants and toddlers and it must be differentiated from other disorders that can have a similar presentation in this age group. JMML is very rare and the diagnosis is often difficult to establish. Some of the genetic variants of JMML may do well without chemotherapy or with minimal chemotherapy, although the majority of patients need a hematopoietic stem cell transplant (HSCT) to achieve cure.

For hematopoietic stem cell transplantation (HSCT), the occurrence of procedure-related deaths needs to be counterbalanced by the reduction in relapse risk. The procedure-related deaths are dependent on the intensity of the prior induction chemotherapy. Despite intensive treatment, ˜30% of the pediatric patients relapse, and outcome is poor, reflected by the ˜30%-40% of patients surviving.

Pre-leukemic conditions, such as myelodysplastic syndromes (MDS) and myeloproliferative disorders (MPDs) including: chronic myelogenous leukemia, polycythemia vera, essential thrombocytosis, agnogenic myelofibrosis and myeloid metaplasia, and others. Antibodies include free antibodies and antigen binding fragments derived therefrom, and conjugates, e.g. pegylated antibodies, drug, radioisotope, or toxin conjugates, and the like.

The types of cancer that can be treated using the subject methods of the present invention include but are not limited to pediatric acute myeloid leukemia, juvenile myelomonocytic leukemia, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

Hematologic cancers are of interest, e.g. leukemias and lymphomas. Acute myeloid leukemia, e.g. pediatric AML is of particular interest.

CD4^{IL-10} cells, also referred to herein as LV-10 cells. See, for example, WO2013192215A1; WO2016146542A1, each herein specifically incorporated by reference. In some embodiments the cytotoxic T cells are CD4+ T cell engineered to produce high levels of IL-10, referred to as LV-10 cells. In particular, an homogenous IL-10-engineered CD4⁺ T (CD4^{IL-10}) cell population has been generated by transducing human CD4+ T cells with a bidirectional lentiviral vector (LV) encoding for human IL-10, leading to a constitutive over expression of IL-10. The CD4^{IL-10} cell population is able to eliminate tumor cells, while maintaining an intrinsic characteristic, Tr1-like, to prevent xeno-GvHD. The CD4^{IL-10} cell population kills tumors (or target cells) expressing CD13. The expression of CD13 on the tumor or target cells is determinant for the anti-tumoral activity of the CD4^{IL-10} cell population. The killing activity of CD4^{IL-10} requires the presence of CD13, HLA-class I, and CD54 on the tumor. The adoptive transfer of CD4^{IL-10} cells mediates in vivo potent anti-tumor effect, e.g. an anti-leukemia effect, and prevents xeno-GvHD without compromising the GvL effect mediated by HSCT. The cells can be allogeneic or autologous, and may be allo-antigen-specific or polyclonal cells.

CD4^{IL-10} cells homogenously express GzB, are CD18⁺, which in association with CDIIa forms LFA-1, CD2⁺, and CD226⁺. Anti-leukemic activity of CD4^{IL-10} cells is specific for myeloid cells and requires the presence of HLA-class I on the tumor.

Cytotoxic T lymphocytes (CTL) are CD8⁺ cells that can be reactive to tumor cells. Induction and expansion of CTL is antigen-specific, and MHC restricted. Various types of cytokines including IL-2 have also been reported to induce cytotoxic lymphocytes.

One class of T lymphocytes with antitumor activity has been termed “tumor-infiltrating lymphocytes” (TIL). They can be grown by culturing single-cell suspensions obtained from tumors in IL-2. Although lymphocytes comprise only a small subpopulation of the cells in a cancer nodule, some of these lymphocytes contain IL-2 receptors and grow under the influence of IL-2. Although tumor cells also grow in the culture, lymphocytes capable of eliminating the tumor cells have a selective growth advantage. After 2-3 weeks of culture, pure populations of lymphocytes without contaminating tumor cells are obtained.

Cytokine-induced killer (CIK) cells are highly efficient cytotoxic effector cells obtained by culturing peripheral blood lymphocytes (PBLs) in the presence of IFN-gamma, IL-2 (or IL-12), and monoclonal antibody (MAb) against CD3, and optionally include IL-Ia. Cells may be cultured for at least about 1 week, at least about 2 week, at least about 3 weeks, or more, and usually not more than about 8 weeks in culture. CIK cells possess a high level of cytotoxic activity.

Cytotoxic T cells for use in the methods as described above may be collected from a subject or a donor. The cells may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxic cells, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintain Tr1 cells the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS) or human serum or serum-free complete media.

The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

The cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection. Optionally the reprogrammed cells are selected for expression of LAG3 and CD49b prior to use.

Expression construct: The coding sequences for knocking out CD200R1, alone or in combination with expression of IL-10, etc. may be introduced on an expression vector into a cell to be engineered. For example, a reprogramming factor coding sequence may be introduced into a target cell using CRISPR technology. CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The nucleic acid encoding a reprograming factor is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors may contain a selection gene, also termed a selectable marker. This gene encodes a protein necessary for the survival or growth of transformed host cells grown in a selective culture medium or a truncated gene encoding a surface marker that allows for antibody based detection. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, or (d) enable surface antibody based detection for isolation via fluoresences activating cell sorting (FACS) or magnetic separation e.g. truncated forms of NGFR, EGFR, CD19.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the ABD construct coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning a construct are the prokaryotic, yeast, or other eukaryotic cells described above. Examples of useful mammalian host cell lines are mouse L cells (L-M[K-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including T cells, stem cells, etc. can be transfected with the above-described expression vectors for construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent, e.g. an infusion of engineered T cells, etc., sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

The cytotoxic T cells and CD200 blocking agent may be used alone or in combination with other therapeutic intervention such as radiotherapy, chemotherapy, immunosuppressant and immunomodulatory therapies, cell therapy, and transplantation.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

### Cell Compositions

In some embodiments a T cell composition is provided in combination with a CD200 blocking agent. The cell can be provided in a unit dose for therapy, and can be allogeneic, autologous, etc. with respect to an intended recipient. Methods may include a step of obtaining desired cells, e.g., T cells, hematopoietic stem cells, etc., which may be isolated from a biological sample, or may be derived in vitro from a source of progenitor cells. The cells are transduced or transfected with a vector comprising a sequence encoding the reprogramming factors, which step may be performed in any suitable culture medium. For example, cells may be collected from a patient, modified ex vivo, and reintroduced into the subject. The cells collected from the subject may be collected from any convenient and appropriate source, including e.g., peripheral blood (e.g., the subject's peripheral blood), a biopsy (e.g., a biopsy from the subject), and the like.

Where the use of autologous cells is not desirable, e.g. where a patient has insufficient T cells for modification, where there is insufficient time to expand autologous cells, etc., allogeneic cells may be used, e.g. T cells or stem cells from a healthy donor.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

Kits may be provided, e.g. including cells or reagents suitable for isolating and culturing cells; reagents suitable for culturing T cells; and reagents. Kits may comprise a CD200 blocking agent. Kits may also include tubes, buffers, etc., and instructions for use.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Does CD200 Inhibit NK Cell Mediated Cytotoxicity Against Cancer Cells?

### Method:

Cell prep. For these studies, primary NK cells or clinically-used human NK-92 cell line (ATCC) are used to perform degranulation and cytotoxicity assays. K562 cells (ATCC) are natural targets of NK cell mediated degranulation and K562-CD200^{hi} cell lines are generated by lentivirally overexpressing CD200 in these cells as performed using U937 and ALL-CM previously described. Healthy donor PBMCs are used for isolation of primary NK cells via StemCell Technologies NK cell negative isolation kit. Primary NK cells are cultured in CellGenix SCGM, containing 10% Human Serum (HS) for 3 days in the presence of IL-2, -15 and -21 before any use in co-culture assays. Human NK-92 cell line are cultured in CellGenix SCGM, containing 20% Fetal Bovine Serum (FBS), in the presence of IL-2.

Degranulation assays. 5×10⁴ primary NK cells or 2×10⁵ NK-92 cells are cultured 1:1 effector:target (E:T) in 96-well V-bottom plates for 6 hours at 37° C. in the presence of human CD107a antibody. Monensin will be added at the end of the first hour of assay. Effector alone wells are used as negative control to detect background degranulation. PMA and ionomycin are used as positive controls that stimulate maximum degranulation in NK cells. K562 (parental cell line that is not modified), K562-CD200 and K562-CD200^{hi} cell lines are used as target cells. Healthy donor PBMCs can be used as a negative control for NK cell degranulation. In blocking experiments, target cells are treated with 20 ug of unconjugated mouse anti-human CD200 antibody or anti-human CD200R1 antibody or isotype control for 15 minutes before the start of the assay. At the end of 6 hr, cells are stained for surface markers CD3 and CD56 for NK cell gating, GranzymeB, LD aqua for live/dead differentiation and CD33 for K562 cells. CD56⁺GranzymeB⁺CD107a⁺ percentage are recorded for analysis via flow cytometry.

Cytotoxicity assays. Same seeding cell numbers for co-culture conditions are used as above, with varying E:T ratios such as 1:1, 5:1 and 10:1. In one set of experiments, target cells are stained with AnnexinV and PI to assess cells going through apoptosis and necrosis upon NK cell response (16 hr). In the other set, remaining target cells in culture are assessed by comparing them to target alone wells and with direct quantification via cell counting beads in flow cytometry. In blocking experiments, target cells are treated with 20 ug of unconjugated mouse anti-human CD200 antibody or isotype control for 15 minutes at before the start of the assay.

CD200 is expected to inhibit NK cell-mediated degranulation when compared to controls and this effect abrogated with the use of anti-CD200 or CD2001 blocking antibody. Similarly, the results of cytotoxicity assays will show that target cells bearing CD200 on the surface will be protected from NK cell-mediated lysis compared to controls.

### Does CD200 Inhibit CD8+ T Cell Mediated Cytotoxicity Against Cancer Cells?

### Methods:

Cell prep: JY cells are HLA-A2⁺ and are therefore compatible with HLA-A2 restricted peptides for assessing CD8+ T cell responses. Generate two JY derived cell lines: CD200 knockout via CRISPR-Cas9 (JY-CD200⁻) and CD200 overexpressing via lentiviral delivery of CD200 cDNA (JY-CD200^{hi}). Validate CD200 expression via flow cytometry. Isolate CD8⁺ T cells and CD3 depleted PBMCs from a healthy HLA-A2 donor. Purchase peptides for CMV-A2 peptide (NLVPMVATV), working concentration of 2 ug/ml.

Degranulation assay: 1) Stain CD3-depleted PBMC with CMV-A2 MHC-class I tetrameric complexes and then pulse with synthesized CMV-A2 peptide in the presence of anti-CD28/49d, anti-CD107a, and monensin for 5 hr with CD200 or CD200R1 blocking antibody or isotype control. Cells are plated with an effector:target ratio of 1:1 and 10⁵ total cells in each well of a 96-well v bottom plate. Stain cells with CD3, CD8, GranzymeB, and LiveDead Aqua and read on a flow cytometer to quantify the number of antigen specific CD8⁺/GranzymeB⁺/CD107a⁺ cells. 2) Repeat the above experiment but instead of using CD3-depleted PBMC as target cells, use CMV-A2 pulsed JY-CD200 cells and JY-CD200^{hi}. Expected Results: Higher frequency of CMV-A2⁺/CD8⁺/GranzymeB⁺/CD107a⁺ cells in the culture conditions with CD200 blocking antibody. Higher frequency of CMV-A2⁺/CD8⁺/GranzymeB⁺/CD107a⁺ cells in culture conditions using JY-CD200 as target cells.

Cytotoxicity assays: Label JY-CD200 cells and JY-CD200^{hi}, separately, with CFSE and pulse with CMV-A2 peptide. After 60 hours quantify the remaining CFSE⁺ cells on a flow cytometer with CountBright beads. Expected Results: Higher absolute number of CFSE⁺ cells remaining in the JY-CD200^{hi} co-culture condition than the JY-CD200 co-culture condition.

### REFERENCES

Atfy M, Ebian H F, Elshorbagy S E, Atteia H H (2015) CD200 Suppresses the Natural Killer Cells and Decreased its Activity in Acute Myeloid Leukemia Patients. J Leuk 3: 190. doi:10.4172/2329-6917.1000190.
Betts, M. R., Brenchley, J. M., Price, D. A., De Rosa, S. C., Douek, D. C., Roederer, M., & Koup, R. A. (2003). Sensitive and viable identification of antigen-specific CD8+ T cells by a flow cytometric assay for degranulation. Journal of immunological methods, 281(1-2), 65-78.
Coles, S., Wang, E., Man, S. et al, CD200 expression suppresses natural killer cell function and directly inhibits patient anti-tumor response in acute myeloid leukemia. Leukemia 25, 792-799 (2011).
Coles, S. J., Hills, R. K., Wang, E. C. Y., Burnett, A. K., Man, S., Darley, R. L., & Tonks, A. (2012). Expression of CD200 on AML blasts directly suppresses memory T-cell function. Leukemia, 26(9), 2148-2151.
Tonks, A., Hills, R., White, P. et al, CD200 as a prognostic factor in acute myeloid leukaemia. Leukemia 21, 566-568 (2007).

### Example 2

New treatments that preserve GvL while preventing GvHD in the treatment of pediatric AML are urgently needed. To address this need, we devised a novel cell therapy with engineered type 1 regulatory T (Tr1) cells, called LV-10, made by lentiviral transduction of IL10 into peripheral CD4⁺ T cells. Tr1 cells are a FOXP3⁻ subset of peripherally inducible regulatory T cells that correlate with induction of peripheral tolerance in transplanted patients and prevent xeno-GvHD in mice. In addition, Tr1 cells directly lyse and kill malignant myeloid cells via perforin and granzyme B. This killing is not dependent on T cell receptor (TCR) engagement and HLA class II antigen presentation, but rather on the target cell expression of HLA class I and several other molecules that facilitate target cell and T cell interaction. Importantly, LV-10 Tr1 cells were shown to kill primary adult AML blasts and to impair leukemia progression in humanized mouse models of AML.

The sensitivity of pAML to Tr1-mediated killing has not been tested. pAML have significant genetic, epigenetic, and molecular differences in comparison to adult AML. Understanding if pAML are also sensitive to Tr1-mediated killing is thus a critical step in LV-10 cell therapy development. Herein, we used LV-10 Tr1 cells to test 23 primary pAML blasts for their sensitivity to killing. We found that over 80% of pAML could be killed by LV-10 cells, with three levels of sensitivity to killing ranging from sensitive, intermediate resistant, and resistant. Sensitive pAML were enriched for gene signatures of leukocyte chemotaxis and expressed mature myeloid markers including CD64 and CD11c, suggesting a more mature phenotype. When analyzed together with the large NCI TARGET pAML dataset, sensitive pAML formed 3 clusters with TARGET samples, including one enriched for pAML samples with FAB M5 acute monocytic leukemia and pAML with MLL rearrangement, while resistant and intermediate resistant pAML clustered with pAML bearing core binding factor translocations inv(16) or t(8;21)(RUNX1-RUNX1T1) cytogenetic abnormalities. In addition, we identified that resistant pAML may evade LV-10 killing by upregulating CD200, which has been associated with poor prognosis of adult AML. Overall, we determine that a majority of pAML are sensitive to killing by LV-10 cells, and that resistance to killing is associated with a loss of mature myeloid signature and upregulation of CD200.

### Materials and Methods

Subjects. De-identified pAML bone marrow aspirates were collected under written informed consent as part of a study approved by the Stanford University Institutional Review Board (IRB) and obtained from the Stanford School of Medicine's Bass Childhood Cancer Center (CA, USA) tissue bank. Patient demographics are listed in Table 1. Human peripheral blood mononuclear cells (PBMC) were obtained from de-identified healthy donors (Stanford Blood Center, CA, USA) in accordance with IRB guidelines.

Cytotoxicity Assays. Killing assay was performed as previously described. Briefly, target cells were co-cultured at a 1:1 effector to target (E:T) ratio for 3 days. For primary pAML, blasts were thawed and incubated for 2 h in complete X-VIVO15 supplemented with IL-3 (20 ng/mL, Peprotech, NJ, USA) and G-CSF (20 ng/ml, Peprotech). After incubation, blasts were co-cultured for 4 days. Surviving cells were enumerated by FACS using CountBright beads (Thermo Fisher, MA, USA). Elimination efficiency was calculated as 1−(number of targets remaining in LV-10 co-culture/number of targets remaining alone) with 2-4 LV cell lines per pAML.

Degranulation was measured as previously described. Briefly, T cells were co-cultured with target cells at a 10:1 E:T ratio with anti-CD107a antibody. After 1 h, brefeldin A (3 μg/ml) and monensin (2 μM) (eBioscience, CA, USA) were added and incubated for 5 h. Cells were stained, fixed, permeabilized (BD Fixation/Permeabilization kit, BD Biosciences), and stained for intracellular granzyme B. Data was analyzed by flow cytometry. For CD200R1 blocking, 25 ug/ml of CD200R1 or isotype antibody was added to T cells for 30 min at 37° C. prior to co-culturing with targets.

RNA-Sequencing (RNA-Seq). Complete computational methods for RNA-Seq processing, analysis, and raw data are available at GEO under accession number GSE140960. For differential gene expression, DESeq2 was used to normalize the counts and perform exploratory analysis (e.g. clustering, principal component analysis). Genes with low expression across all samples, sum(gene)<10 reads, were filtered out before performing differential gene expression. The design matrix was defined as design=˜condition, where the condition variable was composed of the following three levels: sensitive, intermediate resistant, and resistant. Transcripts were hierarchically clustered using Euclidean distance and complete linkage function. The heatmaps were created using ComplexHeatmap v2.0.0. GO terms were collapsed using EnrichmentMap v3.2.1 in Cytoscape v3.8.0. Correlation graph was plotted in R version 4.0.0. Enrichment analysis was performed using a binomial test for a one-tailed p value, and confidence interval (CI) was calculated using Wilson/Brown test.

Statistical Analysis. For the non-RNA-seq-derived data, analysis was performed using GraphPad Prism 7. As applicable, center bars and whiskers represent the mean with standard deviation, or median with range/interquartile range. The data was analyzed using non-parametric tests that do not assume equal variances between groups: Mann-Whitney or Wilcoxon test for groups of 2 (unpaired or paired samples, respectively), and Kruskal-Wallis or Friedman ANOVA with Dunn's post hoc test for >2 groups (independent or dependent samples, respectively). Multiple testing correction was applied. Linear regressions were plotted using linear regression analysis in GraphPad Prism.

Cytokine Secretion. To measure cytokine secretion upon stimulation, 1×10⁵ LV-GFP or LV-10 cells were incubated for 48 h with stimulation by immobilized anti-CD3 (10 μg/mL) and soluble anti-CD28 (1 μg/mL) in a 96-well round-bottomed plate. The levels of secreted IL-4, IL-10, and IFN-γ were determined by ELISA (BD Biosciences). IL-10 to IL-4 ratio was obtained by dividing IL-10 secretion by IL-4 secretion.

NCI TARGET pAML RNA-seq data. We obtained RNA-seq data of 187 pAML patients from the National Cancer Institute (NCI) initiative: Therapeutically Applicable Research to Generate Effective Treatments (TARGET) on childhood cancers at. When compared to TARGET data, Stanford pAML samples were processed following the same NCI guidelines.

Generation of CD200 overexpressing cell lines. CD200 was amplified from CD200 pORF (ABM, Richmond, BC, Canada) then ligated into pLVX-IRES-ZsGreen1 (Takara Bio, Mountain View, Calif., USA) using XhoI and BamHI cut sites. psPAX2 and pVSVG packaging plasmids were co-transfected with pLVX-CD200-IRES-ZsGreen1 into 293T cells to produce virus. Lentivirus was concentrated using the Lenti-X concentrator (Takara Bio). U937 or ALL-CM cells were transduced with pLVX-CD200-IRES-ZsGreen1 or control lentivirus using retronectin with the manufacture's protocol ‘RetroNectin-Bound Virus Infection Method By Centrifugation’ (Takara Bio). 5 days after transduction, cells were stained and CD200⁺GFP⁺ or GFP⁺ cells were sorted by FACS.

### Results

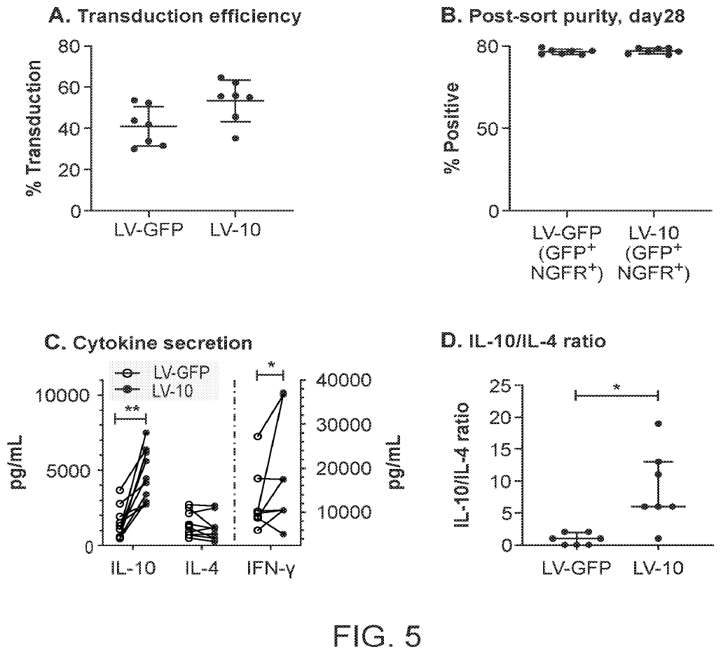

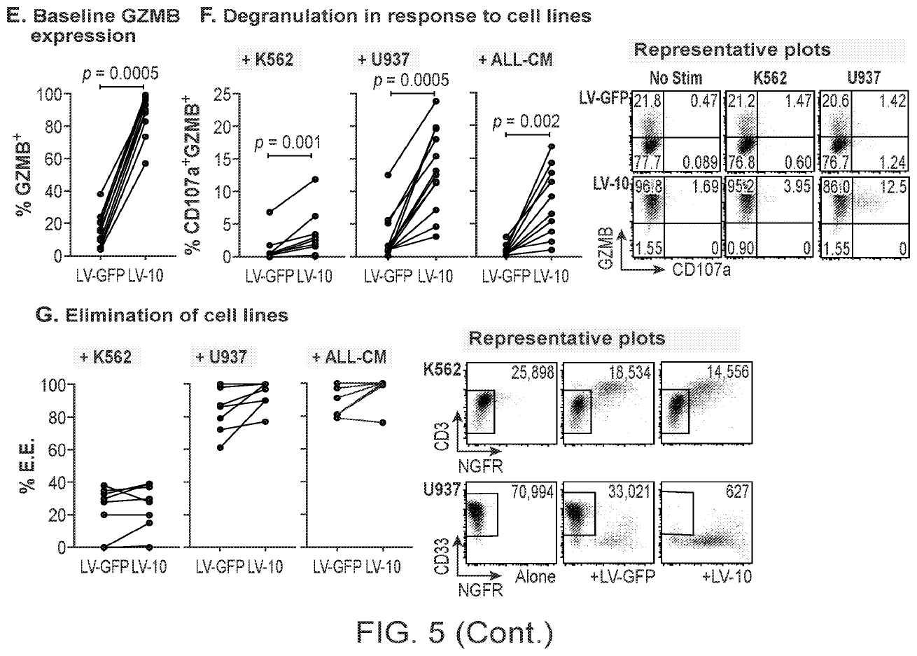

Pediatric AML blasts have different levels of sensitivity to LV-10 killing. To determine if pAML can be killed by LV-10 cells, we first generated LV-10 cells from healthy donor-derived CD4⁺ T cells and verified their transduction efficiency, purity, cytokine profile, and killing capacity (FIG. 5). LV-10 cells had high transduction efficiency, high IL-10 and low IL-4, as well as high intracellular granzyme B expression at baseline (FIG. 5A-E) in comparison with effector T cell (Teff)-like control LV-GFP cells. LV-10 degranulation against target cells was also higher than LV-GFP cells, especially against HLA-class I positive myeloid tumor cell lines U937 and ALL-CM (FIG. 5F). LV-10 cells were able to potently eliminate U937 and ALL-CM cells, but not HLA-class I negative erythroleukemic K562 cell line (FIG. 5G). Target cell elimination was also observed in control LV-GFP cells, which are not tolerogenic and thus are not being further explored for clinical use.

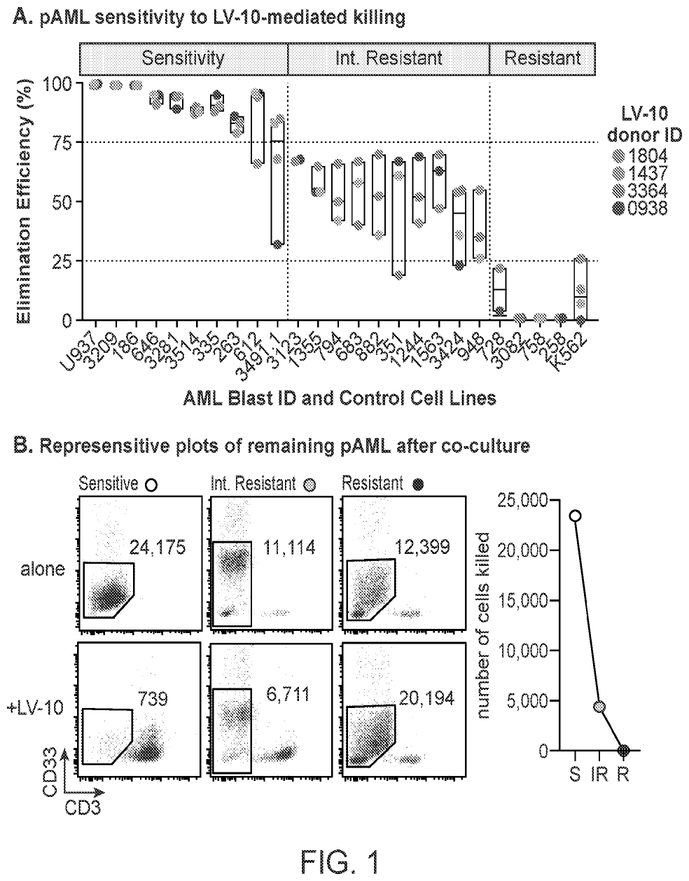

Next, we tested if LV-10 cells could eliminate pAML. We obtained 23 pAML bone marrow aspirates, 18 at onset and 5 at relapse, of various World Health Organization (WHO) and FAB diagnoses (Table 2). Killing-sensitive U937 and -resistant K562 cells were used as positive and negative controls, respectively. In the killing assay (see Materials and Methods), we observed 3 levels of pAML sensitivity to LV-10 killing: sensitive (S, >70% median elimination efficiency, E.E.), intermediate resistant (IR, 25-70% median E.E.), and resistant (R, <25% median E.E.) (FIG. 1A, B). Sensitivity or resistance was retested in 9 pAML samples, and sensitivity levels were reproducible. Notably, all the pAML tested had high levels of HLA class I.

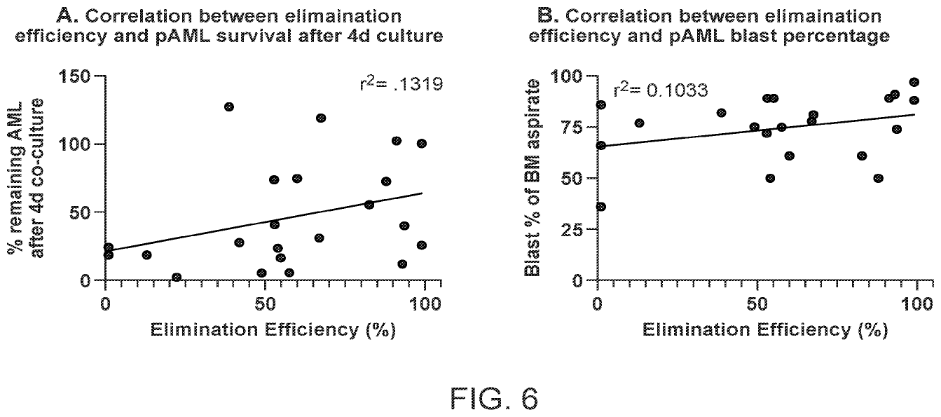

Because primary pAML typically expand poorly in vitro and can undergo spontaneous apoptosis, we examined if sensitivity correlated with pAML survival when cultured without LV-10 cells. Survival of pAML cultured in medium alone did not correlate with their sensitivity to killing when cultured with LV-10 (FIG. 6A). pAML sensitivity to killing also did not correlate with blast percentage within the bone marrow aspirate (FIG. 6B). Notably, pAML sensitivity to killing did not depend on whether the sample was acquired at onset or at relapse. Although our sample set was limited, we observed that 6 of the 7 pAML samples with core-binding factor (CBF) rearrangements (inv(16)(CBFB-MYH11) and t(8;21)(RUNX1-RUNX1T1)), which are associated with a more favorable prognosis, were classified as IR or R.

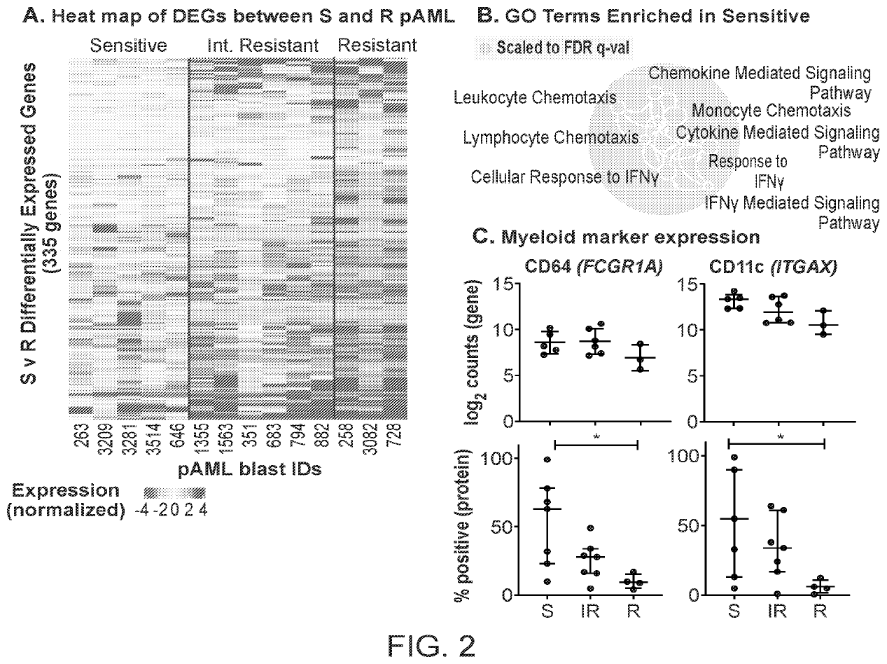

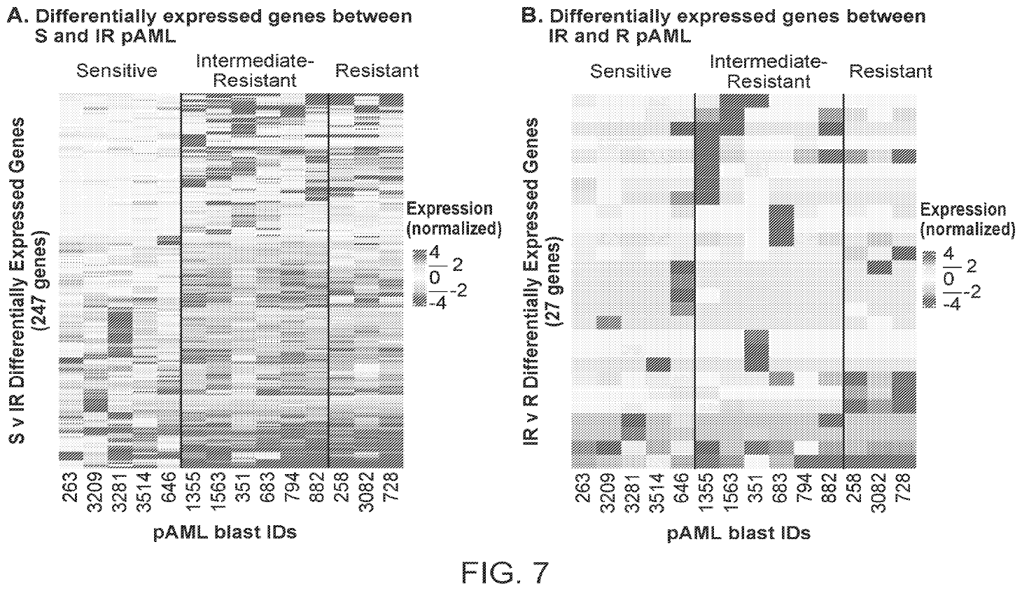

Killing-sensitive pAML have significantly different gene expression than resistant pAML. To identify factors impacting pAML sensitivity to LV-10 killing, we performed RNA-seq on 14 S, IR, and R pAML. We found 335 differentially expressed genes (DEG) between S and R pAML (absolute log 2 fold change (FC) 2, FDR<0.05) (FIG. 2A). Between the other groups, we found 247 DEGs between the S and IR pAML, while the IR and R pAML were more similar, with only 27 DEGs, (FIG. 7A, B).

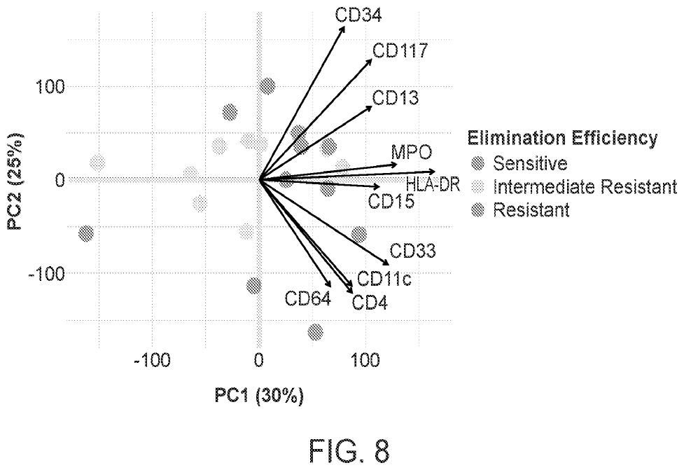

We next examined gene ontology (GO) term enrichment in sensitive and resistant pAML using GSEA. We visualized the results using EnrichmentMap to collapse the GO terms into sub-clusters. Sensitive pAML showed strong signatures of IFN-γ related genes and monocyte chemotaxis (FIG. 2B). We also observed that the protein expression of monocytic genes (CD64, CD11c, CD4, CD15, and CD33) largely contributed to the observed variance amongst the clinical flow cytometry phenotypes of pAML samples (FIG. 8). To investigate this monocytic signature, we visualized the gene expression of selected, established AML maturation markers from the RNA sequencing data (FIG. 2C, top) derived from the bulk bone marrow aspirate lysates, and matched it to the corresponding proteins expressed on pAML blasts, measured by clinical flow cytometry phenotyping (FIG. 2C, bottom). CD11c and CD64 proteins, which are commonly observed in mature, monocytic AML were expressed significantly higher in sensitive than in resistant pAML blasts (FIG. 2C).

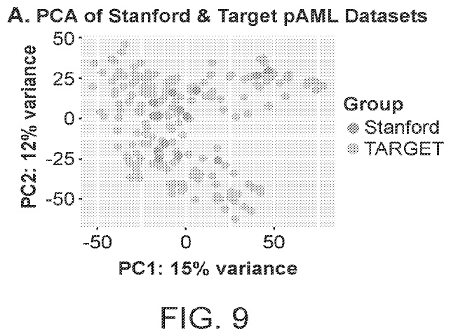

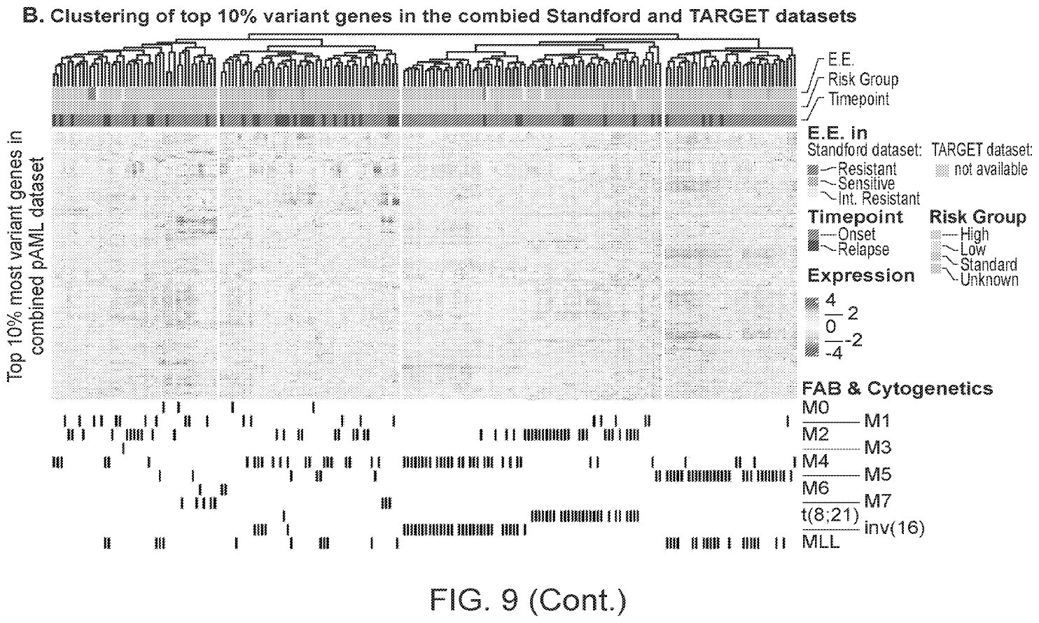

pAML sensitivity and resistance signatures observed in NCI TARGET pAML transcriptome dataset. To determine if the gene expression signatures of sensitivity and resistance we observed in our pAML samples can be found in a larger cohort, we analyzed our dataset together with a 187-sample NCI TARGET pAML dataset, the largest comprehensive pAML dataset publicly available. Principal component analysis on the most variant genes showed that the Stanford pAML samples distributed among the TARGET pAML samples, indicating that the sample source was not a dominant technical covariate (FIG. 9A). Unsupervised analysis of the combined Stanford and TARGET pAML datasets confirmed that Stanford pAML samples did not cluster independently (FIG. 9B). Interestingly, out of the 4 major clusters, 2 clusters contained only the sensitive pAML while the other 2 cluster contained both the intermediate resistant and resistant pAML.

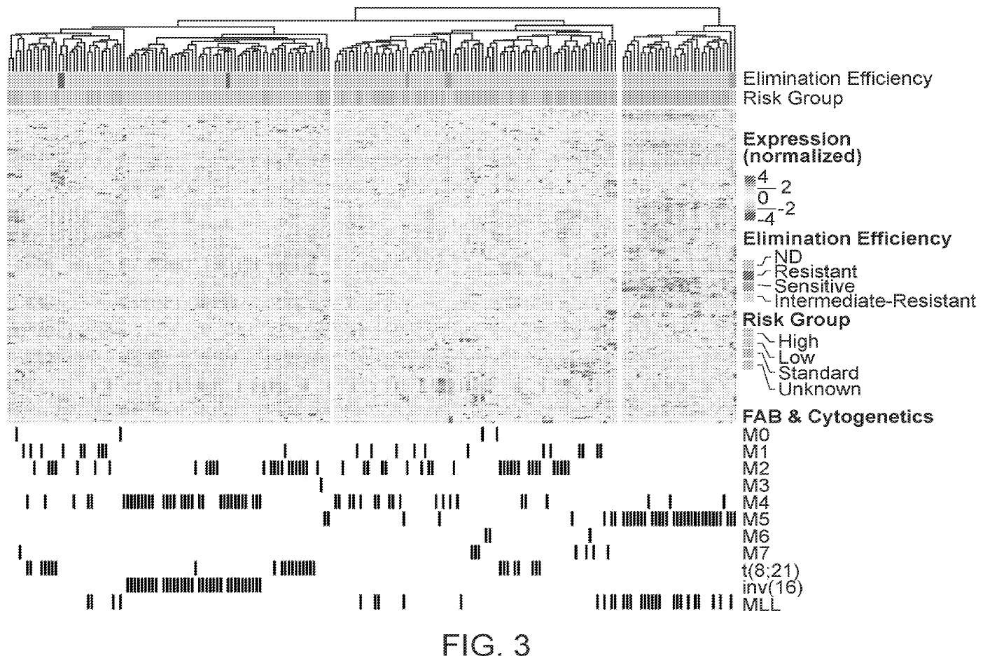

Next, we examined if the 335-gene signature discriminating between S and R pAML was present in the TARGET dataset. Clustering of the combined pAML dataset based on their expression of the identified DEGs grouped the samples into three primary clusters: two ‘sensitive’ clusters that grouped with S pAML and 57% of TARGET pAML, and a ‘resistant’ cluster that grouped with the IR and R pAML and 43% of TARGET pAML (FIG. 3). Because we observed that CBF pAML were highly represented in IR and R pAML, we examined their distribution in the combined Stanford-TARGET dataset. Both pAML with t(8;21)(RUNX1-RUNX1T1) and pAML with inv(16)(CBFB-MYH11) translocations were enriched in the ‘resistant’ cluster (p<0.0001, <0.0001 respectively). In line with our GSEA analysis results, one of the ‘sensitive’ clusters was highly enriched for M5 monocytic pAML (p<0.0001), while the ‘resistant’ cluster was enriched for M4 myelomonocytic pAML that also displayed rearrangement inv(16) (p<0.0001).

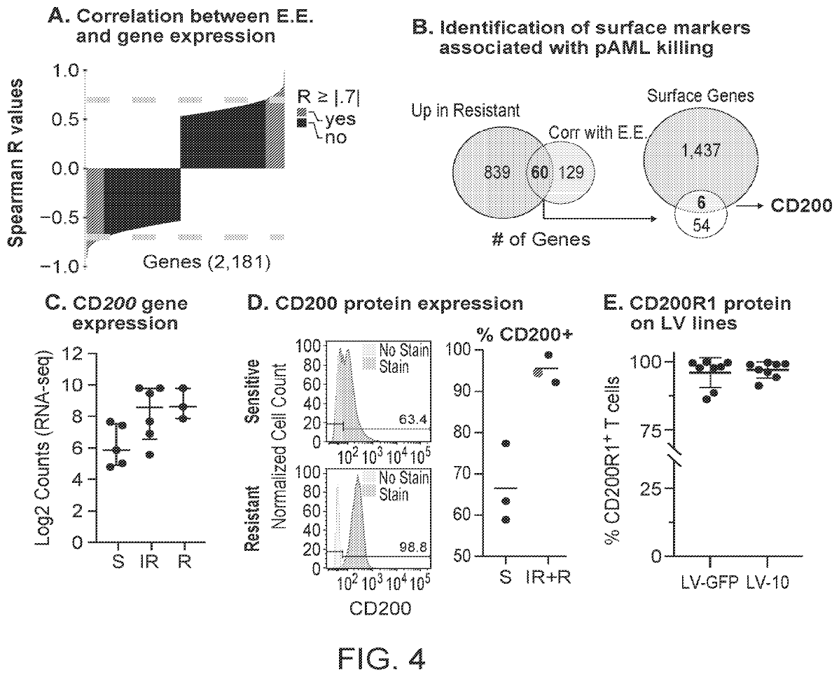

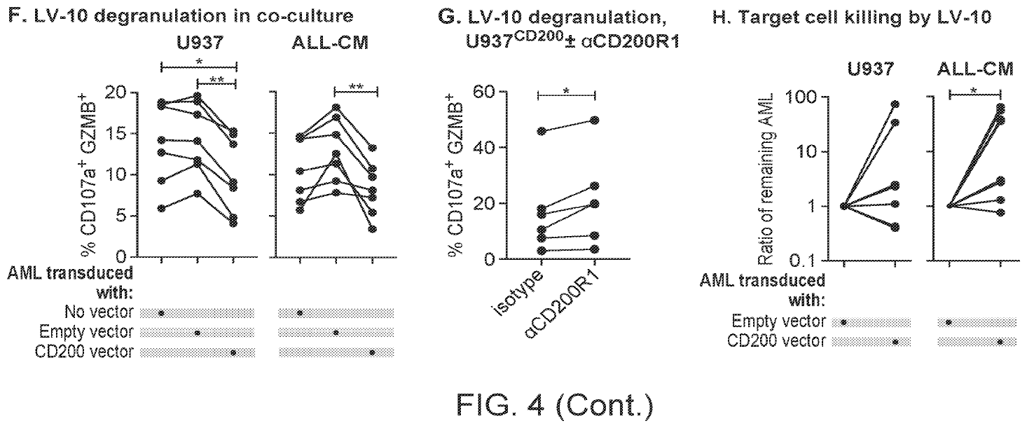

Resistant pAML express high levels of CD200, which can impair LV-10 cytotoxicity. To identify genes linked with pAML sensitivity or resistance to LV-10 cell killing, we correlated gene expression to the median elimination efficiency for each pAML blast. The expression of 2,181 genes significantly correlated to killing with p<0.05 (FIG. 4A), 395 of which had an absolute R ≥0.7 (FIG. 4A, genes shown as grey bars). We hypothesized that the resistant pAML expressed inhibitory markers that protected them from killing. Therefore, we overlaid the genes that significantly and negatively correlated with killing, R≤−0.7 (189 genes) with the genes that were overexpressed 4-fold or more in the resistant pAML from the DEG analysis of sensitive versus resistant pAML (899 genes). We found 60 genes that were both negatively correlated with killing and preferentially expressed in resistant pAML (FIG. 4B). Since perforin and granzyme B-mediated killing requires cell-to-cell interaction, we filtered this gene list for genes encoding surface proteins and identified 10 genes (FIG. 4B).

Next, we manually examined the functions of the 10 genes to uncover proteins that have known interacting receptors expressed on T cells, and identified CD200, a type 1 membrane glycoprotein. CD200 is upregulated on resistant pAML (FIG. 4C, D), and LV-10 express the CD200 receptor CD200R1 (FIG. 4E), an inhibitory receptor of immunoglobulin superfamily. CD200 expression is associated with poor prognosis in adult AML. Moreover, CD200R1 signaling has been previously shown to impair mast cell and CD8⁺ T cell degranulation.

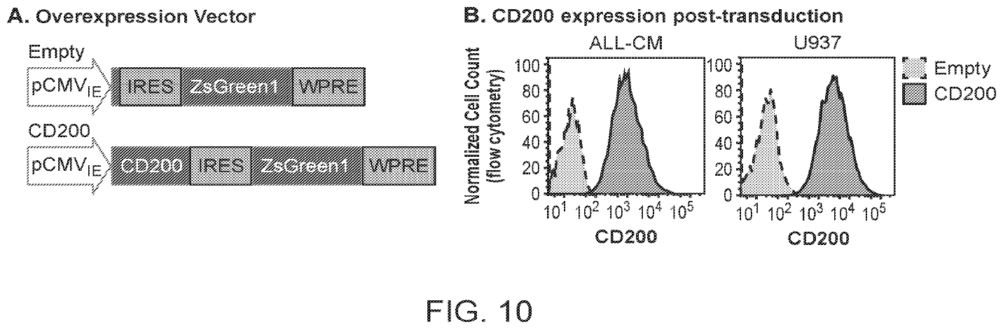

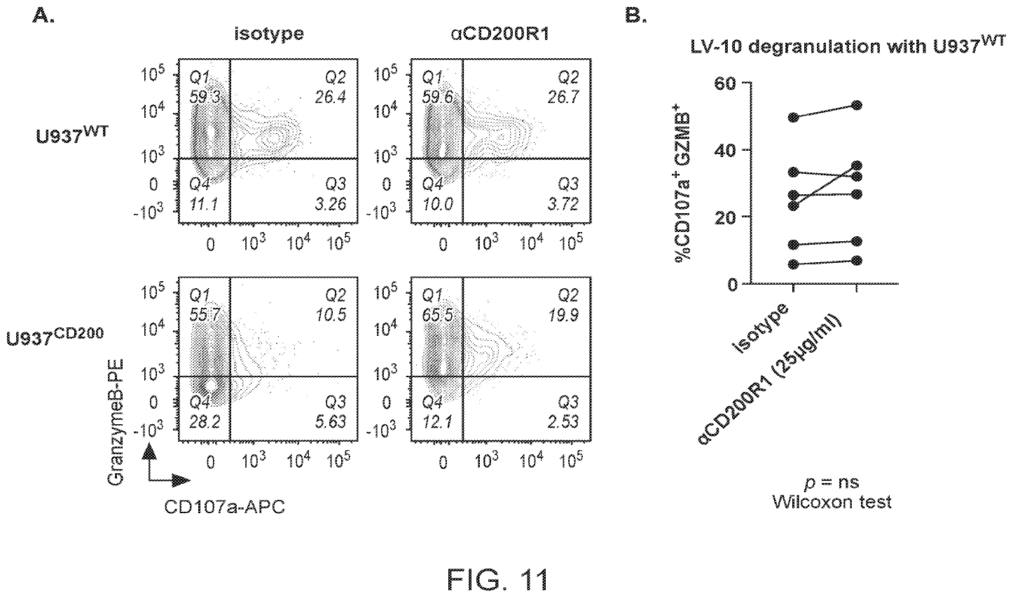

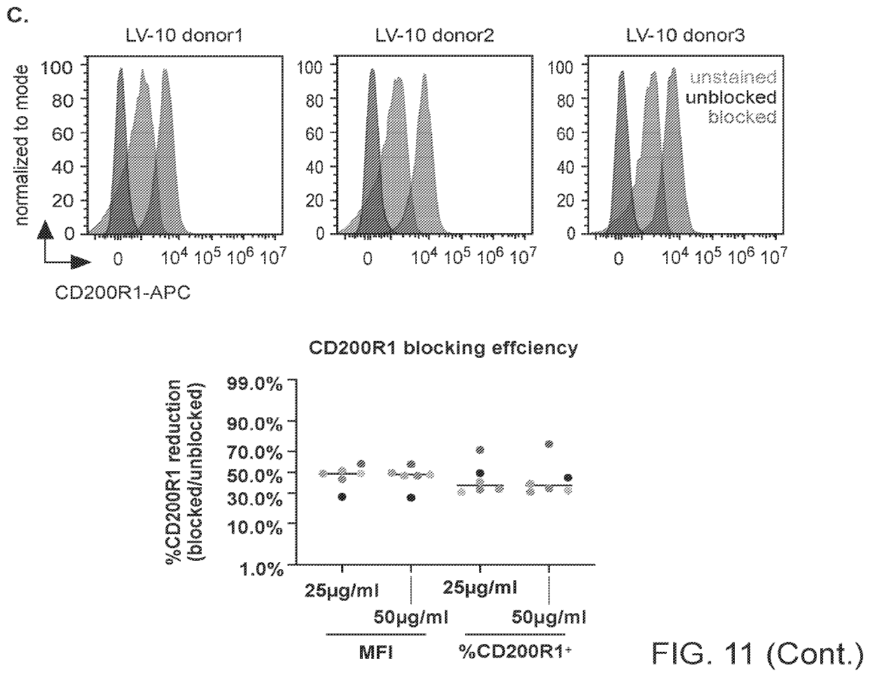

To determine if CD200 expression confers resistance to LV-10-mediated killing, we overexpressed CD200 in killing-sensitive ALL-CM and U937 myeloid cell lines. For this, we constructed a bicistronic lentiviral vector containing CD200 together with ZsGreen1, a green fluorescent protein (FIG. 10A). Both cell lines transduced with the CD200-containing vector displayed significant upregulation of CD200 protein compared to empty vector-transduced cells (FIG. 10B). First, we tested the impact of CD200R1 signaling on LV-10 degranulation using CD107a degranulation assay coupled with granzyme B intracellular staining. In comparison to the LV-10 cells co-cultured with control cell lines, LV-10 co-cultured with CD200-overexpressing cell lines degranulated significantly less (FIG. 4F). To test if we could rescue the reduction in degranulation induced by CD200 overexpressing cells, we blocked CD200R1 on the LV-10 with a neutralizing antibody prior to co-culture with U937 and U937 CD200 overexpressing cell lines (FIG. 11A). Blocking CD200R1 partially restored LV-10 degranulation when co-cultured with CD200 overexpressing U937 (FIG. 4G), while it had a non-significant effect on LV-10 degranulation when co-cultured with wild type U937 (FIG. 11B). LV-10 degranulation was not fully restored to levels induced by wild type U937, likely because the CD200R1 neutralizing antibody only blocked approximately 50% of available CD200R1 (FIG. 11C).

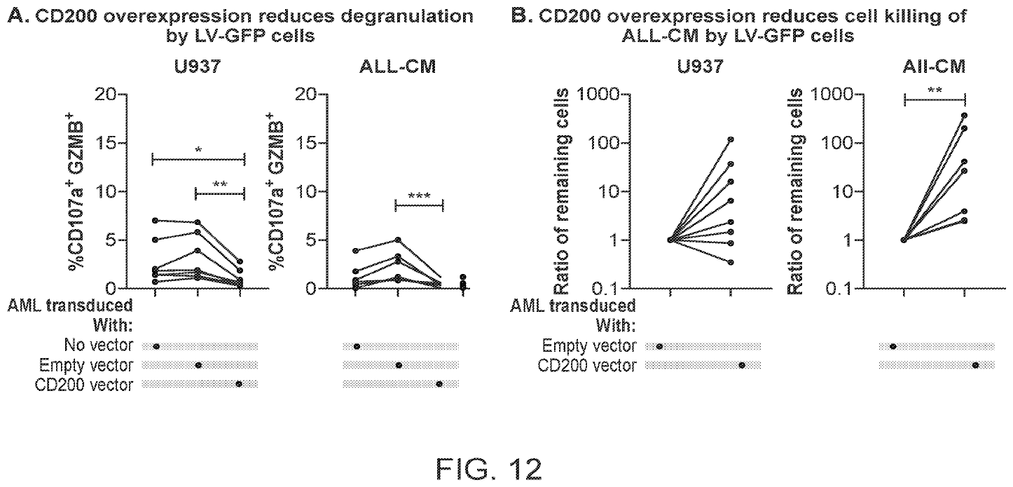

We next tested if CD200 overexpression on myeloid leukemia cell lines could confer resistance to LV-10 killing. In comparison to the empty vector-transduced control cells, CD200 overexpression significantly reduced killing of ALL-CM cells, but not of U937 cells (FIG. 4H). This may be due to U937 cells' increased robustness in vitro, as they have an average 1.34-fold higher proliferation rate than ALL-CM cells (not shown) that could compensate for killing in a 3-day culture. LV-GFP degranulation and killing, which are less potent than in LV-10 cells (FIG. 12), was also impaired by CD200, indicating that the CD200R1 signaling-induced inhibition of cytotoxicity is not Tr1-specific. Altogether, these data suggest that resistant pAML can evade LV-10 killing by impairing their degranulation via CD200 expression. Pediatric AML blasts have different levels of sensitivity to LV-10 killing. To determine if pAML can be killed by LV-10 cells, we first generated LV-10 cells from 4 healthy donor-derived CD4⁺ T cells and verified their transduction efficiency, purity, cytokine profile, and killing capacity. LV-10 cells had high transduction efficiency, high IL-10 and low IL-4, and killed the sensitive U937 myeloid tumor cell line.

AML is a highly diverse hematopoietic cancer with over 20 different WHO sub-classifications, with suboptimal responses to conventional therapy and an urgent need for novel treatments. Our previous study revealed that 4 of 8 primary adult AML were sensitive to LV-10 cell killing. Importantly, LV-10 cells could inhibit myeloid leukemia progression in vivo while preventing the induction of GvHD when co-injected with CD4⁺ T cells, suggesting that LV-10 cells can represent an innovative cell therapy for AML. Since pAML differ substantially from adult AML at the molecular, epigenetic, and genetic levels, herein we determined the pAML sensitivity to LV-10 killing, characterized the sensitive and resistant pAML molecular profiles, and identified CD200 expression as one of the mechanisms of pAML resistance to LV-10 killing.

While previously tested adult AML had only two levels of sensitivity to LV-10 killing, resembling the intermediate resistant and resistant pAML we measured, we also observed a subset of pAML that were highly sensitive to elimination by LV-10 cells. This additional sensitivity category may reflect the intrinsic genetic and epigenetic differences between adult and pediatric AML, which could affect expression of markers required for LV-10-mediated killing. Interestingly, we observed that the expression of CD13, CD54, or CD112, which positively correlated with sensitivity to LV-10-mediated killing in adult AML, did not correlate to pAML sensitivity to killing, further supporting the hypothesis that pediatric and adult AML interact differently with LV-10 cells.

The range of sensitivities we observed in pAML was underscored by significant differences in gene expression and cytogenetics. These analyses revealed that sensitivity to killing was linked to differentiation status. Sensitive pAML resembled more mature differentiated myeloid cells, with an enrichment of monocytic genes and high levels of CD64 and CD11c protein, which are frequently described on more differentiated AML subtypes. Conversely, resistant pAML did not have as distinct a gene signature. We found the maturation signature we observed in our sensitive subset present in the 187-sample NCI TARGET-AML dataset. Whether we clustered the combined Stanford and TARGET data sets using only the top variably expressed genes or with our filtered S v R DEG list, the S pAML independently clustered away from the IR/R pAML. This was partially because the top 10% variability expressed genes in the combined Stanford and TARGET pAML datasets incorporated around half of the 335 DEGs discriminating S v R pAML, yet this also suggests that the S v R DEGs may represent underlying distinguishing features among pAML. In addition to the genes driving the clustering, cytogenetic abnormalities, specifically the core binding factor translocations t(8;21)(RUNX1-RUNX1T1) and inv(16), were consistently overrepresented within the IR and R pAML containing cluster. Despite the typically more favorable prognosis for pAML with core binding factor translocations, these pAML could evade T cell killing in vitro. While the role of core binding factor translocations in immune evasion is not well understood, it has been observed these lesions can impair NK cell surveillance of target cells through downregulation of CD48, and NK cell ligand. Conversely, in the S containing cluster, there was enrichment of patients with MLL rearrangements that historically have intermediate to poor outcome. MLL rearrangements account for 15-20% of all pAML cases, but only 3% of adult AML, which suggest a that LV-10 cells may be uniquely suited for the treatment of a common pAML subset. Further analysis of the sensitivity of specific subsets of pAML to LV-10 mediated killing may improve our ability to identify key genes responsible for the sensitivity of these pAML subsets.

We also identified CD200 as upregulated on IR and R pAML. CD200 has previously been associated with poor patient outcomes in adult AML. CD200 is a membrane glycoprotein that induces an inhibitory signal upon binding to its cognate inhibitory receptor CD200R1, and impairs degranulation in mast cells and CD8⁺ T cells. CD200R1 is expressed on both LV-10 and control LV-GFP cells. CD200 has negligible baseline expression on killing-sensitive ALL-CM, U937, and THP-1 myeloid cell lines. We found that the overexpression of CD200 on ALL-CM and U937 cell lines led to a significant impairment in LV-10 degranulation, and in one cell line, CD200 overexpression also increased AML survival in the killing assay. CD200 effect on LV-10 degranulation was specific to CD200/CD200R1 interaction, as the degranulation increased upon CD200R1 receptor blockade. Interestingly, CD200 expression also impaired the response of the Teff-like control LV-GFP cells. These results, together with reports showing that CD200 expression on AML can impair CD8⁺ T cell function, support the role of CD200 signaling in the impairment of cytotoxic T cell degranulation. Notably, degranulation and killing of CD200-overexpressing AML cell lines were only impaired, not completely abolished, again suggesting that resistant pAML express multiple genes that contribute to their evasion of LV-10 killing.

Our observation that LV-10 cells can eliminate a large subset of pAML, together with our previously published data showing their ability to eliminate AML cell lines in vivo, support their use as a novel therapy for high-risk pAML patients receiving allo-HSCT. Uses of LV-10 cells in the clinic include donor-derived LV-10 cells could be used alongside allo-HSCT, acting early to prevent GvHD and combat residual AML. Alternatively, LV-10 can be used for their GvL effect when the patients' own immune cells are depleted. Patients who are minimal residual disease positive after induction chemotherapy have an abysmal prognosis, with only 10% disease-free survival. In those patients, LV-10 cells could be used as a less toxic alternative to another round of induction chemotherapy prior to allo-HSCT. LV-10 would eliminate residual AML blasts, while persisting 2-3 weeks in vivo without eliciting GvHD, until the patient's own immune system reconstitutes. Notably, to mediate killing, LV-10 cells do not need to recognize specific antigens on their target cells through the TCR, uncoupling their cytotoxicity from HLA-II match or mismatch.

In conclusion, we show that LV-10 cells can directly mediate killing of pAML, especially those with an activated, mature myeloid gene expression profile. pAML resistance to killing was associated with expression of CD200, an immunomodulatory protein associated with poor AML prognosis, which could impair LV-10 cytotoxic responses. By blocking the effect of these resistance factors, we can reverse resistance to LV-10-mediated killing. LV-10 cell therapy is well suited to treat pAML by providing both a GvL effect and preventing GvHD, thus improving the outcome for many children with high risk pAML.

TABLE 1
Patient Demographics

| Age (at sample acquisition) | 137 mo (5-267 mo) |
|---|---|
| % Female | 43.50% |
| Average white count at | 83.7 K/μl (0.4-347.6) |
| Risk Stratification | |
| High Risk | 12/23 |
| Standard Risk | 5/23 |
| Low Risk | 6/23 |
| Average time of follow-up | 33.5 mo |
| Progression to HSCT | 43.50% |
| Overall survival | 73.90% |

TABLE 2
Pediatric AML patient clinical characteristics
Table 2: Characteristics of pediatric AML. pAML samples were grouped based on their
sensitivity to LV-10-mediated killing. Sample timepoint, WHO classification, FAB classification,
cytogenetics, blast percentage, WBC count at diagnosis, age in months, risk group stratification,
and minimal residual disease (MRD) status after first induction chemotherapy are displayed.
WBC, white blood cell.

| AML | Response | Sample | WHO | FAB | | % | WBC | Age | Risk | |
|---|---|---|---|---|---|---|---|---|---|---|
| ID | to LV-10 | Timepoint | Classification | Subtype | Cytogenetics Onset | Blast | (10³/mL) | (M) | group | MRD |
| 3209 | Sensitive | Onset | AML - NOS | M5a | 46, XY, t(X; 11)(5′MLL+) | 97 | 347.6 | 5 | H | − |
| 186 | | Relapse | AML - NOS | M5a | 46, XY, MLL+ | 88 | 51.6 | 156 | H | + |
| 646 | | Relapse | AML with | M4Eo | 46, XY, del(7q)(q22) | 74 | 177 | 267 | H | + |
| | | | MDS-related | | | | | | | |
| 3281 | | Onset | AML - NOS | M5b | 46, XX | 91 | 8.7 | 215 | H | − |
| 3514 | | Onset | AML - NOS | M2 | 51, XY, +X, +9, +11, +14, +20 | n/a | 4 | 157 | S | + |
| 335 | | Onset | AML - NOS | M5a | 46, XY, FLT3-ITD+ | 89 | 174 | 35 | H | − |
| 263 | | Onset | AML with mutated | AML | 46, XY | 61 | 9.8 | 141 | H | + |
| | | | RUNX1 | w/MDS | | | | | | |
| 612 | | Onset | t(8; 21); | M2 | 46, XY, t(8; 21) | 50 | 39.9 | 205 | L | − |
| | | | RUNX1-RUNX1T1 | | | | | | | |
| 3491.1 | Intermediate- | Onset | APL with | M3 | 46, XX, t(15; 17) | 78 | 4.4 | 138 | S | − |
| | Resistant | | PML-RARA | | | | | | | |
| 3123 | | Relapse | MPAL | MPAL | 46. XX, Complex karyotype: | 81 | 102 | 134 | H | + |
| 1355 | | Onset | APL with | M3 | 46, XY, t(15; 17) | 75 | 2.1 | 119 | S | − |
| | | | PML-RARA | | | | | | | |
| 794 | | Onset | Inv(16); | M2 | 46, XX, Inv(16) | 89 | 43 | 28 | L | + |
| | | | CBFB-MYH11 | | | | | | | |
| 683 | | Onset | Inv(16); | M4Eo | 46, XY, Inv(16) | 89 | 56.1 | 190 | L | − |
| | | | CBFB-MYH11 | | | | | | | |
| 882 | | Onset | MPAL | MPAL | 46, XY, t(7; 14)(g21; q32) | 72 | 180.1 | 176 | H | + |
| 351 | | Onset | t(8; 21); | M2 | 47, XX, t(8; 21), +21 | 75 | 0.4 | 194 | S | + |
| | | | RUNX1-RUNX1T1, | | | | | | | |
| | | | trisomy 21 | | | | | | | |
| 1244 | | Onset | AML with mutated | M7 | 48, XY, +21, +Y | 50 | 37 | 14 | H | + |
| | | | RUNX1, trisomy 21 | | | | | | | |
| 1563 | | Onset | t(8; 21); | M2 | 46, XX, t(8; 21) | 61 | 51.3 | 178 | L | + |
| | | | RUNX1-RUNX1T1 | | | | | | | |
| 3424 | | Relapse | t(9; 11); | M5a | 46, XX, t(9; 11) | n/a | 2.8 | 162 | S | + |
| | | | MLLT3-KMT2A | | | | | | | |
| 948 | | Onset | AML with | M2 | 46, XX, Complex karyotype | 82 | 153.7 | 51 | H | − |
| | | | mutated NPM1 | | | | | | | |
| 728 | Resistant | Onset | AML - NOS | M1 | 46, XY, t(1; 13)(p34~36; | 77 | 3.9 | 207 | H | + |
| | | | | | q13~14)[19] | | | | | |
| 3082 | | Relapse | Inv(16); | M4Eo | 46, XX, Inv(16) | 36 | 283 | 37 | L | − |
| | | | CBFB-MYH11 | | | | | | | |
| 758 | | Onset | Inv(16); | M4Eo | 46, XX, Inv(16) | 66 | 2.7 | 198 | L | − |
| | | | CBFB-MYH11 | | | | | | | |
| 258 | | Onset | MPAL | MPAL | 46, XY | 86 | 190 | 144 | H | − |

Legend:
MRD = minimal residual disease after first induction chemotherapy,
WBC = White blood cell
H = Hgh,
S = Standard,
L = Low risk group

### Example 3

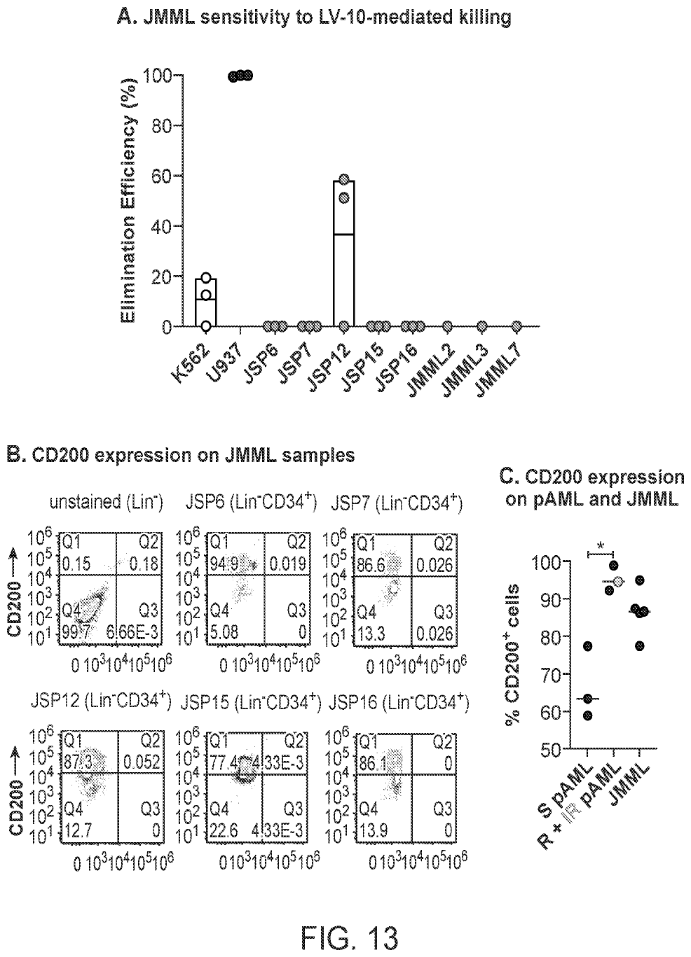

Juvenile myelomonocytic leukemia (JMML) is a clonal hematopoietic disease of early childhood, which shares the features of both myeloproliferative/myelodysplastic malignancies and chronic myelomonocytic leukemias of adulthood. JMML has an estimated survival rate of only ˜50%, and is currently treatable only by hematopoietic stem cell transplantation. We tested eight primary JMML patient samples for their sensitivity to LV-10 cell killing; seven samples were 100% resistant to LV-10 cell-mediated killing, while one sample was intermediate resistant (FIG. 13A). This JMML resistance to LV-10 killing is likely caused by their universally high expression of the CD200 protein (FIG. 13B), which is comparable to the resistant and intermediate resistant pediatric AML samples (FIG. 13C).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for treating cancer with a cytotoxic immune cell, the method comprising: administering an effective dose of a cytotoxic immune cell composition in combination with an effective dose of a CD200 blocking agent that blocks CD200 from interacting with its receptor expressed on the immune cells.

2. The method of claim 1, wherein the cancer is a myeloid leukemia.

3. The method of claim 2, wherein the myeloid leukemia is AML.

4. The method of claim 3, wherein the AML is pediatric AML.

5. The method of claim 2, wherein the myeloid leukemia is juvenile myelomonocytic leukemia (JMML).

6. The method of claim 1, wherein the cytotoxic immune cell is an NK cell.

7. The method of claim 1, wherein the cytotoxic immune cell is a T cell.

8. The method of claim 6, wherein the T cell is a CD8+ T cell.

9. The method of claim 1, wherein the cytotoxic immune cell is an engineered CD4+ T cells expressing IL-10.

10. The method of claim 1, wherein the CD200 blocking agent binds to CD200.

11. The method of claim 10, wherein the agent is an antibody.

12. The method of claim 1, wherein the CD200 blocking agent binds to CD200R1.

13. The method of claim 1, wherein the cytotoxic immune cells are engineered to reduce expression of CD200R1.

14. The method of claim 1, wherein the cancer cells are assessed for expression of CD200 prior to treatment.
